(19) United States
(12) Patent Application Publication
(10) Pub. No.: US 2009/0163431 A1
(43) Pub. Date: Jun. 25, 2009

(54) COMPOSITIONS AND METHODS FOR MODULATION OF PDX-1

(75) Inventors: Irene Kazhdan, San Antonio, TX (US); Zhiqiang Han, Fenton, MO (US); Zheng Jim Wang, San Antonio, TX (US); Ting-Tung Chang, San Antonio, TX (US); Robert A. Marciniak, San Antonio, TX (US)

Correspondence Address: GIFFORD, KRASS, SPRINKLE,ANDERSON & CITKOWSKI, P.C, PO BOX 7021, TROY, MI, 48007-7021, US

(73) Assignee: Ontherex LLC, San Antonio, TX (US)

(21) Appl. No.: 12/031,139

(22) Filed: Feb. 14, 2008

Related U.S. Application Data
(60) Provisional application No. 60/889,808, filed on Feb. 14, 2007.

(51) Int. Cl. A61K 31/7088; C12N 5/06; C12N 15/00

(52) U.S. Cl. 514/44; 435/375; 435/320.1

(57) ABSTRACT

Methods and compositions for inhibiting PDX-1 are provided according to the present invention. An anti-PDX-1 agent included in inventive methods and compositions includes an antibody, an aptamer, an antisense oligonucleotide, a ribozyme and/or an inhibitory compound. Methods of inhibiting PDX-1 expression in a tumor cell are provided by the present invention which include contacting a tumor cell with an effective amount of an anti-PDX-1 agent highly or completely complementary to a specified region of an RNA molecule encoding PDX-1. Such an agent specifically hybridizes with the RNA molecule encoding PDX-1 and inhibits the expression of a PDX-1 gene in the tumor cell. Compositions including anti-PDX-1 siRNA and/or shRNA are described. Recombinant expression constructs encoding anti-PDX-1 siRNA or shRNA according to the present invention are described.

### REFERENCE TO RELATED APPLICATIONS

This application claims priority of U.S. Provisional Patent Application 60/889,808, filed Feb. 14, 2007, which is incorporated herein by reference in its entirety.

### FIELD OF THE INVENTION

The present invention relates generally to compositions and methods for modulation of PDX-1. In particular, the present invention relates to compositions and methods for inhibiting expression and/or activity of PDX-1 in tumor cells as a therapy for cancer.

### BACKGROUND OF THE INVENTION

Pancreas duodenal homeobox-1 (PDX-1) is a homeodomain-containing transcription factor important in the embryonic development of normal pancreas and in insulin activation in pancreatic cells. During embryonic development, PDX-1 is expressed in all pancreatic cells, although expression is restricted to beta and delta cells in adults. Nucleotide and amino acid sequences for PDX-1 have been characterized. FIG. 1 illustrates the structure of the PDX-1 protein which includes a transactivation domain, a homeodomain and a nuclear localization sequence (NLS). An mRNA encoding PDX-1 has been sequenced and is shown and referred to herein as SEQ ID No. 1. The primary amino acid sequence encoded by the coding sequence of SEQ ID No. 1 is shown and referred to herein as SEQ ID No. 2. Mature PDX-1 is a 283 amino acid protein with a molecular weight of 31 kDa.

PDX-1 has a critical role in beta cells of the normal pancreas. Homozygous inactivation of PDX-1, that is, 100% loss of PDX-1 activity, results in pancreas agenesis and animal death shortly after birth. Heterozygous inactivation of PDX-1, causing 50% loss of PDX-1 activity, results in type 2 diabetes in the affected animal. PDX-1 is believed to maintain beta cell function in a normal animal and regulates beta cell proliferation and neogenesis/differentiation. While PDX-1 has a critical role in pancreatic cells of adults as well as in differentiating animals, most normal animal tissues, including most normal human tissues, do not appear to express PDX-1.

In addition to its normal role in healthy tissue, PDX-1 has recently been implicated in cell and tissue pathology apart from diabetes, particularly in cancer. FIG. 2 shows PDX-1 overexpression in human cancer cells, suggesting that dysregulation of PDX-1 expression might be relevant to tumor pathologies. However, there are currently no methods or compositions for modulating PDX-1 expression in order to address abnormal PDX-1 overexpression and inhibit tumor cells characterized by PDX-1 overexpression.

There is a continuing need for methods and compositions for modulating PDX-1 expression for use in treatment and research relating to proliferative disorders, including cancers.

### SUMMARY OF THE INVENTION

Methods of inhibiting a tumor cell are provided according to the present invention which include contacting a tumor cell with an anti-PDX-1 agent. The anti-PDX-1 agent inhibits PDX-1 activity and thereby inhibits the tumor cell. In particular embodiments of methods of the present invention, an anti-PDX-1 agent inhibits expression of PDX-1. For example, the anti-PDX-1 agent is a double-stranded RNA compound that inhibits expression of a PDX-1 gene by RNA interference in certain embodiments of the invention.

A double-stranded RNA compound that inhibits expression of a PDX-1 gene by RNA interference includes about 32 to about 60 nucleotides, an antisense strand and a sense strand, wherein the antisense strand and the sense strand each includes about 16 to about 30 nucleotides. The antisense strand includes a nucleotide sequence having 12 to about 26 sequential nucleotides which is complementary to a nucleotide sequence in the sense strand which includes at least about 12 to about 26 nucleotides. In particular embodiments, the double-stranded RNA compound is assembled from synthesized or expressed unconnected antisense strands and highly complementary sense strands.

In further embodiments of the present invention, a double-stranded anti-PDX-1 RNA compound includes an antisense strand and a sense strand connected by a linker. Non-limiting examples of linkers include an oligonucleotide linker, a polynucleotide linker and a non-nucleotide linker. In particular embodiments, an oligonucleotide linker having from 1-S nucleotides is used.

An anti-PDX-1 agent included in inventive methods can also be an antibody, an aptamer, antisense oligonucleotide, a ribozyme or an inhibitory compound, such as an organic or inorganic inhibitory compound.

In preferred embodiments where an anti-PDX-1 agent includes an antisense oligonucleotide, the antisense strand is substantially complementary to a nucleic acid molecule encoding a human PDX-1. In specific examples, the antisense strand is substantially complementary to a nucleic acid selected from the group consisting of: SEQ ID No. 3, SEQ ID No. 4, SEQ ID No. 5, and SEQ ID No. 6. In further specific examples, the antisense RNA oligonucleotide strand includes SEQ ID No. 8, SEQ ID No. 9, SEQ ID No. 10, or SEQ ID No. 11.

Methods of inhibiting PDX-1 expression in a tumor cell are provided by the present invention which include contacting a tumor cell with an effective amount of an anti-PDX-1 agent highly or completely complementary to a specified region of an RNA molecule encoding PDX-1. Such an anti-PDX-1 agent specifically hybridizes with the RNA molecule encoding PDX-1 and inhibits the expression of a PDX-1 gene in the tumor cell. In particular embodiments of the invention, the anti-PDX-1 agent is directed to a specified region of a nucleic acid molecule encoding human PDX-1 (SEQ ID No. 2).

Particular anti-PDX-1 agents include an antisense nucleic acid sequence highly or completely complementary to SEQ ID No. 3. SEQ ID No. 4, SEQ ID No. 5, or SEQ ID No. 6. For example, the antisense RNA oligonucleotide strand includes SEQ ID No. 8, SEQ ID No. 9, SEQ ID No. 10, or SEQ ID No. 11. In certain embodiments the antisense RNA oligonucleotide strand is identical to SEQ ID No. 8, SEQ ID No. 9, SEQ ID No. 10, or SEQ ID No. 11 and optionally further includes 1-4 additional bases at the 3′ end.

Optionally, contacting a tumor cell with an anti-PDX-1 agent is achieved by specific delivery of the anti-PDX-1 agent to the tumor cell such as direct administration to a tumor and/or including a targeting element with the anti-PDX-1 agent.

A second anti-cancer therapeutic agent and/or an anti-cancer treatment is optionally administered to inhibit a tumor cell.

Compositions are provided according to the present invention which include an anti-PDX-1 agent. Optionally, a pharmaceutically acceptable carrier is included in inventive compositions.

Particular compositions of the invention include an antisense oligonucleotide directed to a specified region of a nucleic acid molecule encoding PDX-1. The antisense oligonucleotide specifically hybridizes with the nucleic acid molecule encoding PDX— and inhibits the expression of PDX-1 in the tumor cell.

Specific compositions include an antisense nucleic acid sequence highly or completely complementary to SEQ ID No. 3, SEQ ID No. 4, SEQ ID No. 5, or SEQ ID No. 6. For example, the antisense RNA oligonucleotide strand includes SEQ ID No. 8, SEQ ID No. 9, SEQ ID No. 10, or SEQ ID No. 11. In certain embodiments of composition of the present invention, the antisense RNA oligonucleotide strand is identical to SEQ ID No. 8, SEQ ID No. 9, SEQ ID No. 10, or SEQ ID No. 11 and optionally further includes 1-4 additional bases at the 3′ end.

An anti-PDX-1 agent included in a composition of the present invention is optionally conjugated to a cell targeting moiety. For example, a cell targeting moiety conjugated to an anti-PDX-1 agent specifically targets the anti-PDX-1 agent to a tumor cell. A second therapeutic agent is optionally included in a composition of the present invention.

Recombinant expression constructs are provided by the present invention which encode an anti-PDX-1 agent including an antisense RNA anti-PDX-1 agent. In particular embodiments, recombinant expression constructs are provided by the present invention which encode an siRNA strand or strands and/or an shRNA effective to inhibit PDX-1 expression in a cell. In a further particular embodiment, recombinant expression constructs are described encoding SEQ ID No. 8, SEQ ID No. 9, SEQ ID No. 10, or SEQ ID No. 11 alone or in combination with SEQ ID No. 17, SEQ ID No. 18, SEQ ID No. 19 or SEQ ID No. 20, respectively. The recombinant expression constructs optionally encode a 1-4 base 3′ overhang at the 3′ end of these or other encoded anti-PDX-1 nucleic acid sequences. Recombinant expression constructs are described including SEQ ID No. 22, SEQ ID No. 23 and/or or SEQ ID No. 24 which encode anti-PDX-1 shRNAs of the present invention.

DNA sequences encoding shRNA directed to PDX-1 targets are anti-PDX-1 agents in particular embodiments of methods and compositions of the present invention. For example, DNA sequences encoding anti-PDX-1 shRNA include SEQ ID No. 22, SEQ ID No. 23 and SEQ ID No. 24.

An isolated mammalian cell line which is homozygous or heterozygous for a mutation preventing expression of a PDX-1 protein is described according to the present invention. Further described is an inventive non-human animal, such as a mouse, including genetically engineered somatic and germ cells which are homozygous or heterozygous for a mutation preventing expression of a PDX-1 protein.

### BRIEF DESCRIPTION OF THE DRAWINGS

FIG. 1 is a diagrammatic representation of the pancreas duodenal homeobox-1 protein including indication of particular domains of the protein;

FIG. 2 is a graph illustrating PDX-1 overexpression in human cancer cells assessed by quantitative PCR;

FIG. 3 is a diagrammatic representation of a lentiviral vector including an shRNA expression cassette driven by human U6 promoter;

FIG. 4 is a graph illustrating suppression of PDX-1 mRNA in MiaPaCa2 pancreatic cancer cells transfected with PDX-1 siRNA compared to cells transfected with control siRNA, as determined by quantitative real-time PCR;

FIG. 5A is a graph of quantified analysis of an immunoblot illustrating decreased PDX-1 levels in MiaPaCa 2 pancreatic cancer cells infected with a lentiviral vector expressing anti-PDX-1 siRNA, but not with a lentiviral vector expressing control siRNA;

FIG. 5B is a digitized image of an immunoblot illustrating decreased PDX-1 levels in MiaPaCa 2 pancreatic cancer cells infected with a lentivirus expressing anti-PDX-1 siRNA, but not with a lentivirus expressing control siRNA;

FIG. 6A is an image of Western blot demonstrating suppression of PDX-1 protein in MiaPaCa-2 pancreatic cancer cells transfected with anti-PDX-1 siRNA as opposed to control siRNA;

FIG. 6B is an image of Western blot demonstrating suppression of PDX-1 protein in SK-BR-3 breast cancer cells transfected with anti-PDX-1 siRNA compared to untransfected cells or cells transfected with control siRNA;

FIG. 7A is a digitized image illustrating pancreatic cancer cell line UK-PAN-1 after infection with a lentiviral vector expressing control shRNA;

FIG. 7B is a digitized image illustrating cell death of pancreatic cancer cell line UK-PAN-1 after infection with a lentiviral vector carrying an anti-PDX-1 shRNA cassette;

FIG. 7C is a digitized image illustrating cells of HCC 1937 breast cancer cells after infection with a lantiviral vector expressing control shRNA;

FIG. 7D is a digitized image illustrating the cell death of HCC 1937 breast cancer cells after infection with a lentiviral vector carrying an anti-PDX-1 shRNA cassette;

FIG. 7E is a digitized image illustrating the absence of toxicity of a lentiviral vector expressing an anti-PDX-1 shRNA in normal mouse embryonic fibroblast cell line 3T3;

FIG. 7F is a digitized image illustrating the absence of toxicity of a lentiviral vector expressing control siRNA in normal mouse embryonic fibroblast cell line 3T3;

FIG. 8A is a digitized image illustrating pancreatic cancer cell line UK-PAN-1 after transfection with synthetic control siRNA;

FIG. 8B is a digitized image illustrating cell death of pancreatic cancer cell line UK-PAN-1 after transfection with synthetic anti-PDX-1 siRNA;

FIG. 8C is a digitized image illustrating prostate cancer cell line LN-CAP after transfection with synthetic control siRNA;

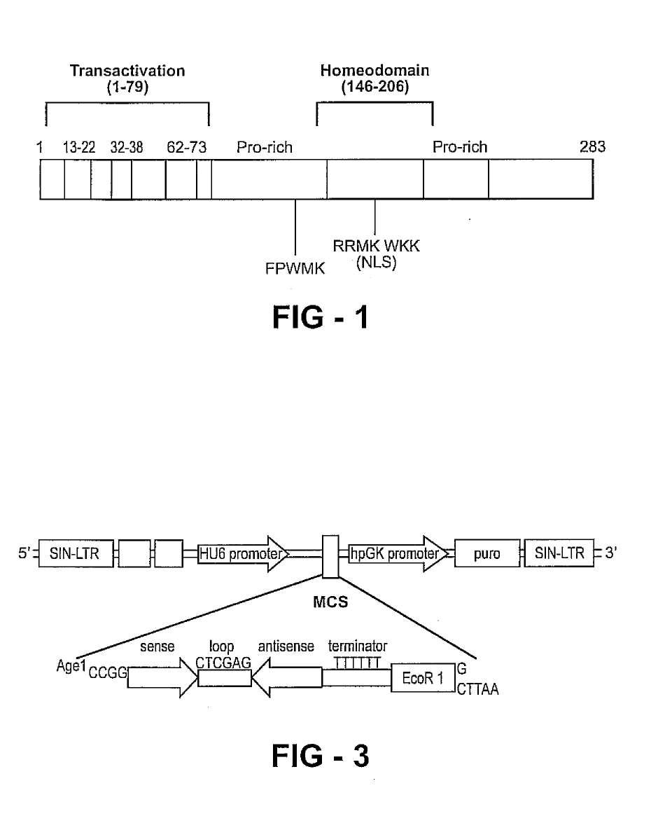

FIG. 5D is a digitized image illustrating cell death of prostate cancer cell line LN-CAP after transfection with synthetic anti-PDX-1 siRNA;

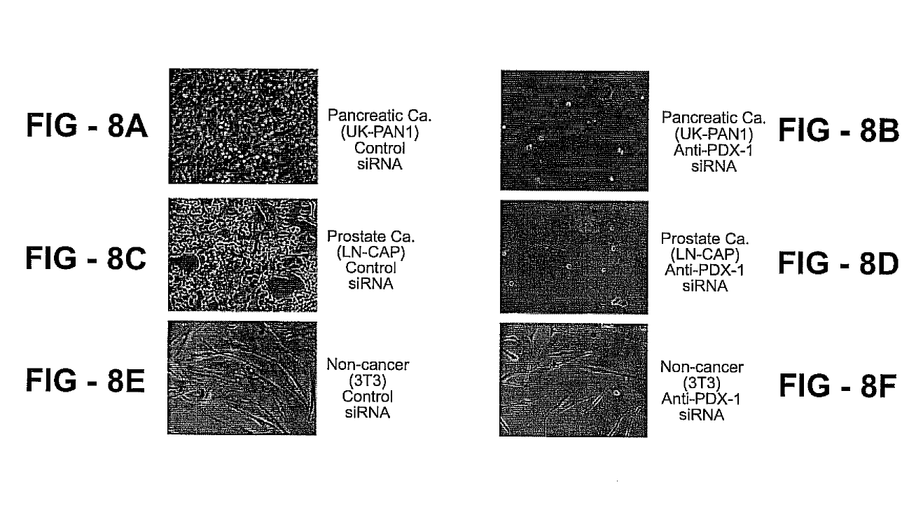

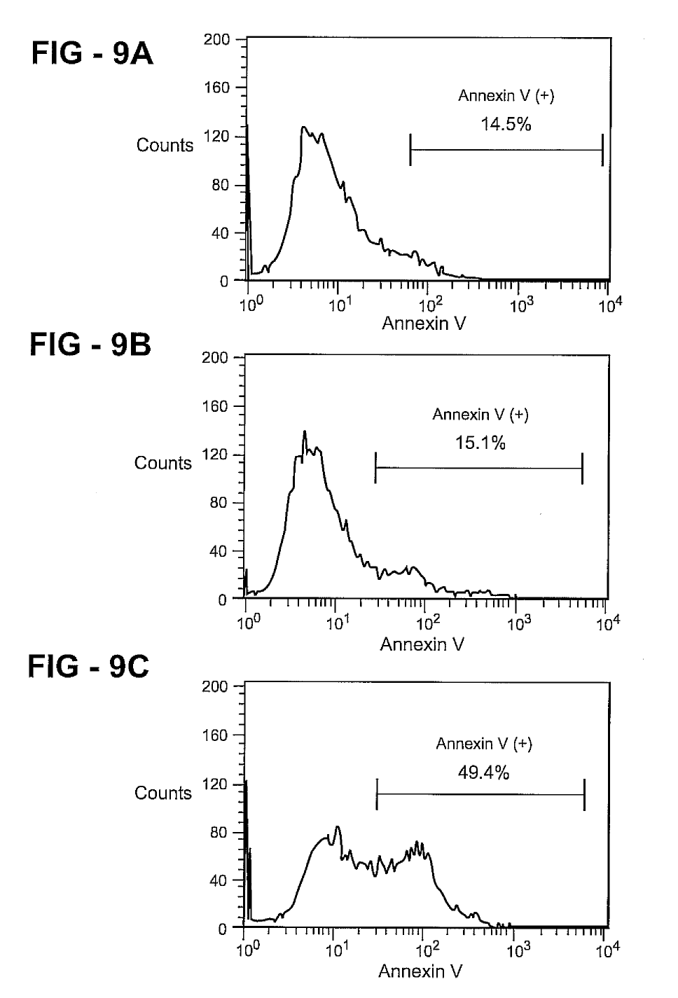

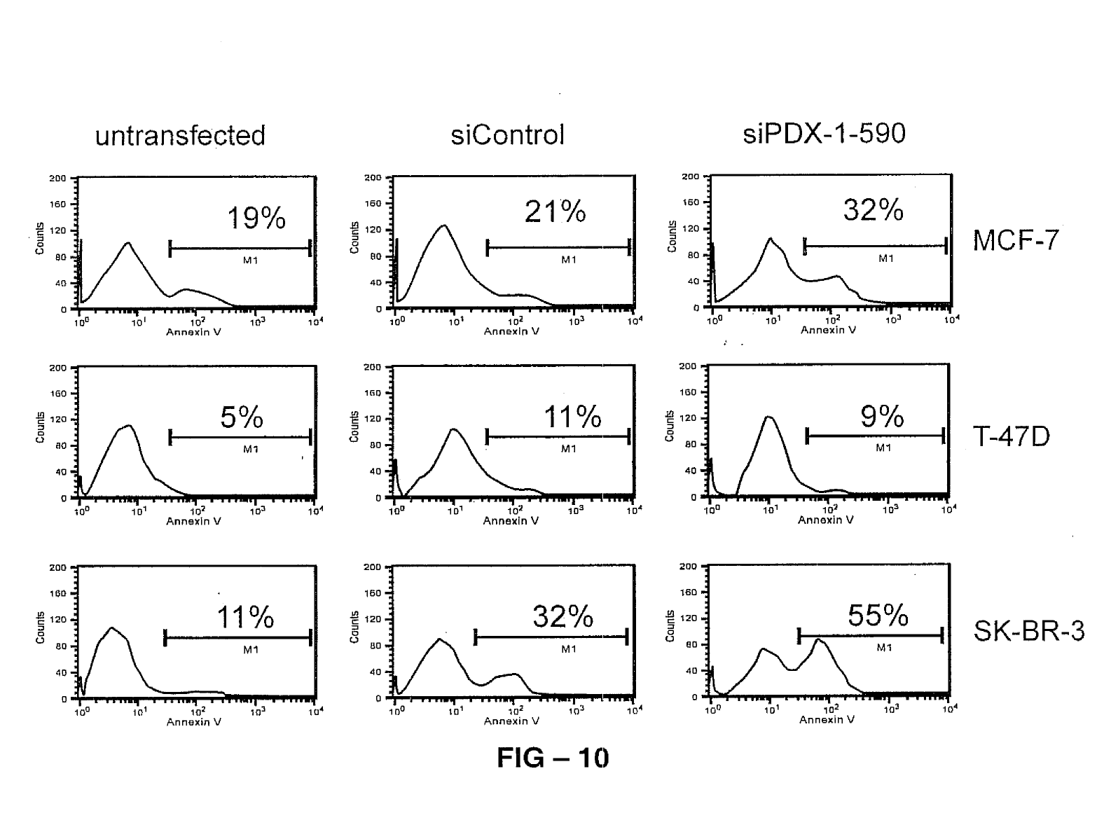

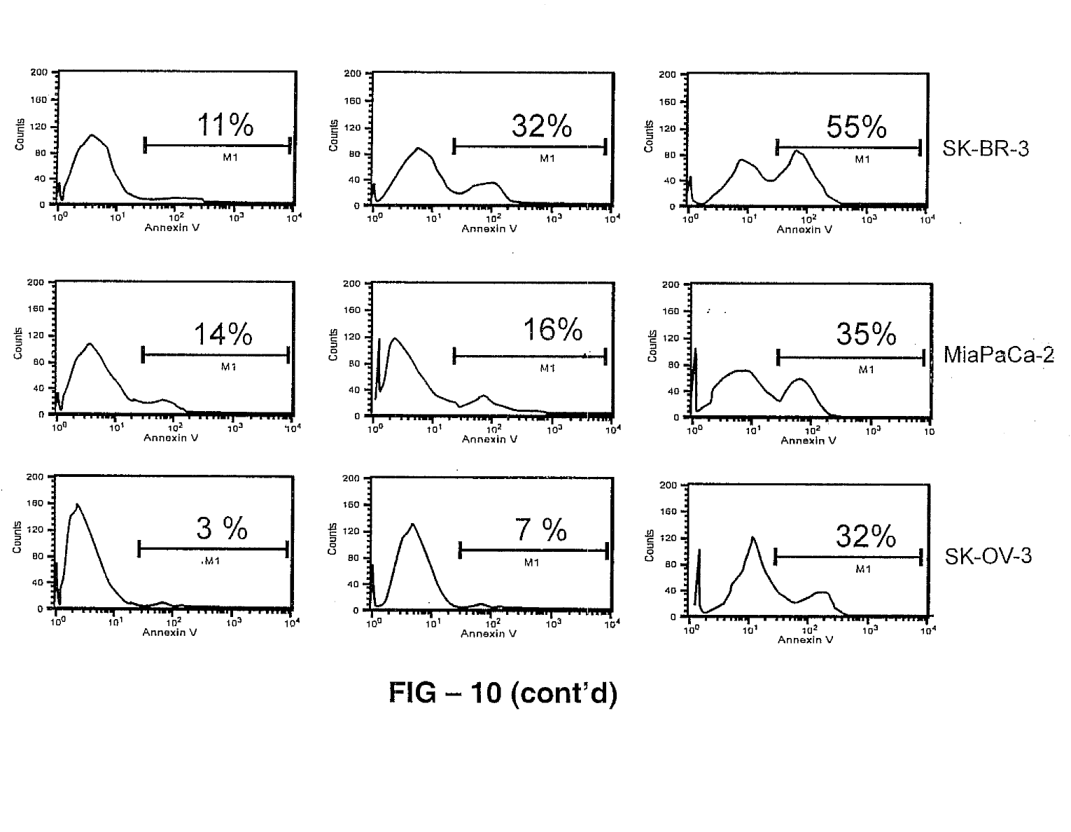

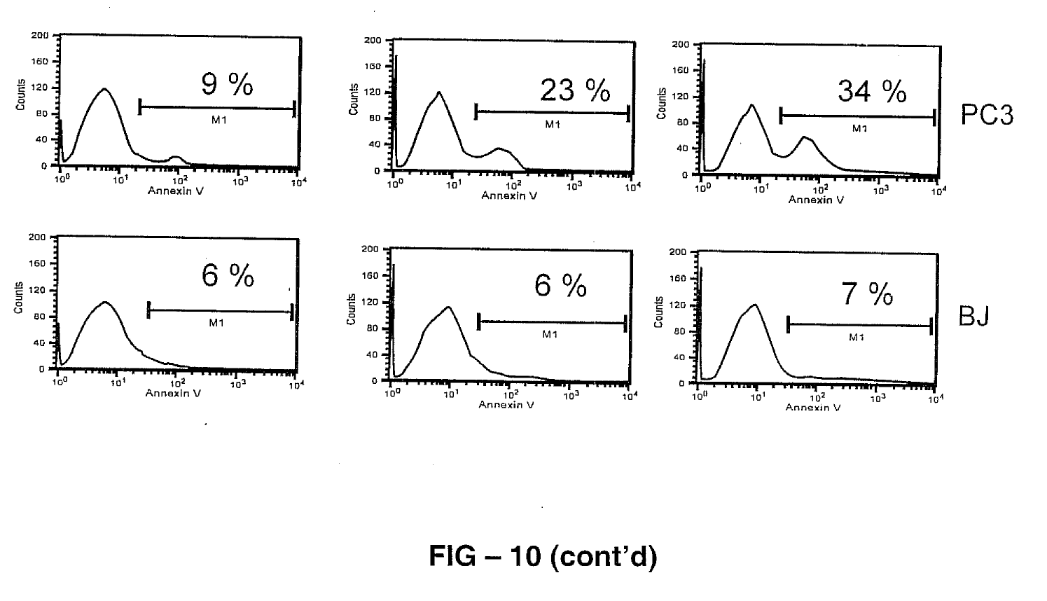

FIG. 8E is a digitized image illustrating the absence of toxicity to normal mouse embryonic fibroblast cell line 3T3 after transfection with synthetic control siRNA anti-PDX-1 siRNA;

FIG. 8F is a digitized image illustrating the absence of toxicity to normal mouse embryonic fibroblast cell line 3T3 after transfection with synthetic compared to cells transfected with synthetic anti-PDX-1 siRNA;

FIG. 9A is a graph illustrating a low percentage of Annexin V positive cells in a population of untransfected MiaPaCa pancreatic cancer cells as analyzed by flow cytometry;

FIG. 9B is a graph illustrating a low percentage of Annexin V positive cells in a population of MiaPaCa pancreatic cancer cells transfected with control siRNA as analyzed by flow cytometry;

FIG. 9C is a graph illustrating a higher percentage of Annexin V positive cells in a population of MiaPaCa pancreatic cancer cells transfected with an anti-PDX-1 siRNA as analyzed by flow cytometry; and

FIG. 10 is a graph illustrating induction of apoptosis identified by binding of FITC-labeled Annexin V and quantified with flow cytometry in cancer cell lines of different tissue origin and absence of apoptosis in normal human fibroblasts after transfection with synthetic anti-PDX-1 siRNA.

### DETAILED DESCRIPTION OF THE INVENTION

A method of treating a tumor is provided which includes delivering to a tumor cell an effective amount of an anti-PDX-1 agent. In addition, compositions are provided according to the present invention which include an anti-PDX-1 agent and a pharmaceutically acceptable carrier. Compositions and methods according to the present invention are believed to be useful in treating a tumor in a subject as well as for inhibiting tumor cells in vitro.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 41th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2006; L. V. Allen, Jr. et S., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 11th Ed., 2005.

The term “tumor” is used herein in its broadest sense and refers to neoplastic growth of cells of various types, illustratively including, but not restricted to squamous cell carcinoma; basal cell carcinoma; transitional cell carcinoma; adenocarcinoma; gastrinoma; cholangiocellular carcinoma; hepatocellular adenoma; hepatocellular carcinoma; renal cell carcinoma; melanoma; fibrosarcoma; myxosarcoma; liposarcoma; leiomyosarcoma; rhabdomyosarcoma; teratoma; hemangiosarcoma; Kaposi sarcoma; lymphangiosarcoma; bone osteoma; osteosarcoma; osteogenic sarcoma; chondrosarcoma; meningioma; non-Hodgkin lymphoma; Hodgkin lymphoma; and leukemia.

An effective amount of an anti-PDX-1 agent is an amount which inhibits activity of PDX-1. In addition, an effective amount is an amount which has the further effect of inhibiting a tumor by stimulating apoptosis in a contacted tumor cell.

An anti-PDX-1 agent inhibits PDX-1 activity in any of various ways including by inhibition of PDX-1 transcription, translation, or transport. Further, inhibition of PDX-1 activity includes inhibition of the functional activities of PDX-1, which includes but is not limited to the inhibition of transcription factor activity of the PDX-1 protein.

An anti-PDX-1 agent is identified by in vitro and in vivo assays including assessment of the effects of a putative anti-PDX-1 agent on PDX-1 mRNA levels, PDX-1 protein levels and/or PDX-1 activity. Assays include RT-PCR, Northern blot, immunoblot, immunoprecipitation, ELISA, assay of PDX-1 binding to target sequences, assay of transcription products of genes for which PDX-1 is a transcription factor, and assays for induction of apoptosis in cells contacted with a putative anti-PDX-1 agent. Illustrative examples of assays for induction of apoptosis include assays for DNA fragmentation, such as electrophoretic detection of nucleosomal ladders, analysis of cell morphology, flow cytometry detection of cell stains indicative of apoptosis, modulation of expression of genes involved in regulation and/or execution of apoptosis and cell cycle and assessment of mitochondrial function.

Anti-PDX agents include nucleic acid agents and protein or peptide agents effective to inhibit PDX-1. An anti-PDX-1 agent also includes agents which are non-nucleic acid, non-protein and non-peptide agents and which are organic or inorganic PDX-1 inhibitory compounds.

The term “nucleic acid” as used herein refers to RNA or DNA molecules having more than one nucleotide in any form including single-stranded, double-stranded, oligonucleotide or polynucleotide. The term “nucleotide sequence” is used to refer to the ordering of nucleotides in an oligonucleotide or polynucleotide in a single-stranded form of nucleic acid.

The terms “duplex” and “double-stranded” are used to refer to nucleic acids characterized by binding interaction of complementary nucleotide sequences. A duplex includes a “sense” strand and an “antisense” strand. Such duplexes include RNA/RNA, DNA/DNA or RNA/DNA types of duplexes. A duplex may be formed from two nucleotide sequences which are otherwise unconnected. Alternatively, a duplex may be formed by a single-stranded nucleic acid where the single-stranded nucleic acid has substantially complementary sense and antisense regions. Such a nucleic acid forms a “hairpin” conformation when the substantially complementary sense and antisense regions are hybridized to form a duplex.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′. It will be recognized by one of skill in the art that two complementary nucleotide sequences include a sense strand and an antisense strand.

The degree of complementarity, also called homology, between nucleic acid strands significantly affects binding of the strands to each other. An antisense strand which is substantially complementary to a sense strand hybridizes to the sense strand under high stringency hybridization conditions.

The term “hybridization” refers to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols, 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The term “specific hybridization” refers to hybridization of a particular nucleic acid to a target nucleic acid without substantial hybridization to nucleic acids other than the target nucleic acid in a cell, tissue or subject.

The term “oligonucleotide” is used herein to describe a nucleotide sequence having from 2-100 linked nucleotides, while the term “polynucleotide” is used to describe a nucleotide sequence having more than 100 nucleotides.

The term “nucleotide” is also used herein as a noun to refer to individual nucleotides or varieties of nucleotides as opposed to a nucleotide sequence.

Illustrative examples of nucleic acid-based agents include antisense molecules such as antisense oligonucleotides and polynucleotides; catalytic nucleic acid-based agents, such as ribozymes; and nucleic acid-based aptamers.

### Nucleic Acid Anti-PDX-1 Agents

In particular embodiments, methods and compositions are provided in which an anti-PDX-1 agent includes an antisense nucleic acid directed to a PDX-1 target sequence. An antisense nucleic acid which is an anti-PDX-1 agent includes a sequence of nucleotides that is highly complementary to a PDX-1 target sequence.

An anti-PDX-1 agent which is a nucleic acid can be produced by chemical synthesis and/or using molecular biology techniques known in the art. For example, chemical synthesis of oligonucleotides is described in Herdewijn, P. (Ed.), Oligonucleotide Synthesis: Methods and Applications, Methods in Molecular Biology, Humana Press, 2004. Molecular biology methods relating to anti-PDX-1 nucleic acid synthesis are described, for example, in Sambrook, J. and Russell, D. W., Molecular Cloning: A Laboratory Manual, Cold Spring Harbor Laboratory Press, 3rd Ed., 2001; and Engelke, D. R., RNA Interference (RNAi): Nuts and Bolts of RNAi Technology, DNA Press LLC, Eagleville, Pa., 2003. Naturally occurring or modified nucleotides may be used in constructing an anti-PDX-1 nucleic acid. Modified nucleotides may be used to increase the stability of an anti-PDX-1 nucleic acid, increase resistance to nucleases, or enhance stability of binding to a target, for instance. Examples of modified nucleotides include phosphorothioates, phosphorodithioates boronophosphates, alkyl phosphonates such as methyl phosphonates, and phosphoramidates such as 3′-amino phosphoramidates.

Generally, antisense nucleic acids useful for inhibiting PDX-1 expression are in the range of about 12 to about 100 nucleotides in length, or longer.

In one embodiment, an anti-PDX-1 agent is a double-stranded RNA molecule that inhibits expression of a PDX-1 gene by RNA interference.

RNA interference is a target sequence-specific method of inhibiting a selected gene. RNA interference has been characterized in numerous organisms and is known to be mediated by a double-stranded RNA, also termed herein a double-stranded RNA compound. Briefly described, RNA interference involves a mechanism triggered by the presence of small interfering RNA, siRNA, resulting in degradation of a target complementary mRNA. siRNA is double-stranded RNA which includes a nucleic acid sequence complementary to a target sequence in the gene to be silenced. The double-stranded RNA may be provided as a long double-stranded RNA compound, in which case it is subject to cleavage by the endogenous endonuclease Dicer in a cell. Cleavage by Dicer results in siRNA duplexes having about 21-23 complementary nucleotides in each of the sense strand and the antisense strand, and optionally 1-2 nucleotide 3′ overhangs on each of the two strands.

Alternatively, siRNA is provided as a duplex nucleic acid having a sense strand and an antisense strand, wherein the sense and antisense strands are substantially complementary and each of the sense and antisense strands have about 16-30 nucleotides. The complementary sense and antisense strands and optionally include 1-2 nucleotide 3′ overhangs on one or both of the two strands. In one embodiment, an siRNA is preferred which has sense and antisense strands, wherein each of the two strands has 21-23 nucleotides, wherein 2 nucleotides on the 3′ end of each strand are overhanging and the remaining 19-21 nucleotides are 100% complementary. As noted above, further details of siRNA compounds are described in Engelke, D. R., RNA Interference (RNAi): Nuts and Bolts of RNAi Technology, DNA Press LLC, Eagleville. PA, 2003. Additional description of siRNA length and composition is found in Elbashir, S. M. et al., Genes and Devel., 15:188-200, 2001; and O'Toole, A. S. et al., RNA, 11:512-516, 2005.

siRNA provided as a duplex nucleic acid having a sense strand and an antisense strand may be configured such that the sense strand and antisense strand form a duplex in hybridization conditions but are otherwise unconnected. A double-stranded siRNA compound may be assembled from separate antisense and sense strands. Thus, for example, complementary sense and antisense strands are chemically synthesized and subsequently annealed by hybridization to produce a synthetic double-stranded siRNA compound.

Further, the sense and antisense strands for inclusion in siRNA may be produced from one or more expression cassettes encoding the sense and antisense strands. Where the sense and antisense strands are encoded by a single expression cassette, they may be excised from a produced transcript to produce separated sense and antisense strands and then hybridized to form a duplex siRNA. See, for example, Engelke, D. R., RNA Interference (RNAi): Nuts and Bolts of RNAi Technology, particularly chapters 5 and 6, DNA Press LLC, Eagleville, Pa., 2003 for further details of synthetic and recombinant methods of producing siRNA.

In a further alternative, a double-stranded “short hairpin” RNA compound, termed “shRNA” or “hairpin siRNA” includes an antisense strand and a sense strand connected by a linker. shRNA may be chemically synthesized or formed by transcription of a single-stranded RNA from an expression cassette in a recombinant nucleic acid construct. The shRNA has complementary regions which form a duplex under hybridization conditions, forming a “hairpin” conformation wherein the complementary sense and antisense strands are linked, such as by a nucleotide sequence of about 1-20 nucleotides. In general, each of the complementary sense and antisense strands have about 16-30 nucleotides.

As noted, siRNA and shRNA may be expressed from a double-stranded DNA template encoding the desired transcript or transcripts. A double-stranded DNA template encoding the desired transcript or transcripts is inserted in a vector, such as a plasmid or viral vector, and operably linked to a promoter for expression in vitro or in vivo. Plasmids and viral vectors suitable for transcription of a double-stranded DNA template are known in the art. Particular viral vectors illustratively include those derived from adenovirus, adeno-associated virus and lentivirus.

The terms “expression construct” and “expression cassette” as used herein refer to a double-stranded recombinant DNA molecule containing a desired nucleic acid coding sequence encoding an siRNA or shRNA and containing appropriate regulatory elements necessary or desirable for the transcription of the operably linked coding sequence in vitro or in vivo. The term “regulatory element” as used herein refers to a nucleotide sequence which controls some aspect of the expression of nucleic acid sequences. Exemplary regulatory elements illustratively include an enhancer, an internal ribosome entry site (“IRES”), an origin of replication, a polyadenylation signal, a promoter, a transcription termination sequence, and an upstream regulatory domain, which contribute to the replication, transcription, post-transcriptional processing of a nucleic acid sequence.

The term “operably linked” as used herein refers to connection of two or more nucleic acid molecules, including an oligonucleotide or polynucleotide to be transcribed and a regulatory element such as a promoter or an enhancer element, which allows transcription of the oligonucleotide or polynucleotide to be transcribed.

The term “promoter” as used herein refers to a DNA sequence operably linked to a nucleic acid sequence to be transcribed such as a nucleic acid sequence encoding an siRNA or shRNA. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce siRNA or shRNA, and provides a site for specific binding by RNA polymerase and other transcription factors.

The term “recombinant” is used to indicate a nucleic acid construct in which two or more nucleic acids are linked and which are not found linked in nature.

As will be recognized by one of skill in the art, particular siRNAs may be of different size and still be effective to inhibit a target gene. Routine assay may be performed to determine effective size and composition of particular compounds. Without wishing to be bound by theory, it is believed that at least the antisense strand is incorporated into an endonuclease complex which cleaves the target mRNA complementary to the antisense strand of the siRNA.

Administration of long RNA duplexes processed to siRNA, as well as administration of siRNA or shRNA, and/or expression constructs encoding siRNA or shRNA, results in degradation of the target PDX-1 mRNA and inhibition of expression of the protein encoded by the PDX-1 mRNA, thereby inhibiting activity of the encoded PDX-1 protein in the cell.

Further details of RNA interference mechanisms as well as descriptions of target identification, synthetic siRNA and shRNA production, siRNA and shRNA expression construct production, and protocols for purification and delivery of expression constructs and synthetic siRNA and shRNA in vitro and in vivo are described in Engelke, D. R., RNA Interference (RNAi): Nuts and Bolts of RNAi Technology, DNA Press LLC, Eagleville, Pa., 2003.

A nucleic acid anti-PDX-1 agent is directed to a specified target sequence of a nucleic acid molecule encoding human PDX-1. A nucleic acid sequence encoding a human PDX-1 protein of SEQ ID No. 2 may be identical to the nucleotide sequence shown in SEQ ID NO. 1, or, owing to the degeneracy of the genetic code, a different nucleic acid sequence may encode the same PDX-1 protein of SEQ ID No. 2. Thus, a nucleic acid anti-PDX-1 agent is directed to a specified target sequence of the nucleic acid sequence of SEQ ID No. 1 or a target sequence of an alternate nucleic acid sequence encoding the PDX-1 protein of SEQ ID No. 2.

A nucleic acid anti-PDX-1 agent may also be directed to a nucleic acid sequence encoding a variant of the protein shown in SEQ ID NO. 2. Variants include naturally occurring allelic variants or non-naturally occurring allelic variants. Such naturally occurring and non-naturally occurring variants include proteins having amino acid deletions, substitutions and additions, as well as fragments, derivatives or analogs of SEQ ID No. 2. The terms “allelic variant,” “fragment,” “derivative” and “analog” when referring to the protein of SEQ ID No. 2 refer to a protein which retains essentially the same biological function or activity as the protein of SEQ ID No. 2.

A PDX-1 target sequence for a nucleic acid anti-PDX-1 agent is identified using several criteria to optimize the PDX-1 inhibiting effects. Target PDX-1 sequences for siRNA and shRNA mediated inhibition are selected which have about a 30-50% GC content. Thus, for example, a 21 nucleotide target sequence optimally includes about 5-11 guanine and/or cytosine residues.

Selected target sequences are compared to other sequences in a nucleic acid database to identify target sequences having significant homology to non-target sequences which may be present in a cell or organism to which the anti-PDX-1 agent is delivered. Such databases may include the publicly available GENBANK, described in Benson D. A., et al., GenBank, Nucleic Acids Res., 2006 Jan. 1; 34(Database issue):D16-20, for instance. Any such identified non-specific sequences are eliminated from consideration for use in methods or compositions according to the present invention. Comparison of target sequences to database sequences may be accomplished using any of various comparison methods and tools known in the art. A commonly used tool is the Basic Local Alignment Search Tool (BLAST), first described in Altschul, S. F. et al., Basic local alignment search tool, J. Mol. Biol., 215:403-10, 1990. The BLAST tools are available for use online or download from the National Center for Biotechnology Information (NCBI) at http://www.ncbi.nlm.nih.gov. One of skill in the art can determine appropriate parameters for detecting homology between a potential target for an anti-PDX-1 nucleic acid agent and other nucleic acid sequences without undue experimentation. For example, for each siRNA, the full nucleotide target sequence was tested for homology with any DNA or RNA sequence in all the databases using BLAST. No known genes other than PDX-1 with significant homology to the target sequences of siRNAs were identified.

Target sequences SEQ ID No. 3, SEQ ID No. 4, SEQ ID No. 5, and SEQ ID No. 6 are identified as targets which are identical to regions of a nucleic acid encoding human PDX-1, SEQ ID No. 1, and which have no significant homology with other human nucleic acid sequences.

An identified target sequence may be further validated by producing an anti-PDX-1 nucleic acid compound directed to the target sequence and assaying the efficacy of the compound in inhibiting PDX-1 transcription and/or translation in vitro and/or in vivo.

An anti-PDX-1 nucleic acid compound, including an siRNA, shRNA or recombinant vector encoding an siRNA or shRNA is introduced into a cell by any of various methods known in the art. For example, a nucleic acid is introduced into a cell via calcium phosphate or calcium chloride co-precipitation-mediated transfection, DEAE-dextran-mediated transfection, liposome-mediated transfection, electroporation and microinjection. Additionally, as noted above, an anti-PDX-1 nucleic acid compound may be introduced into a cell using a viral vector such as those derived from adenovirus, adeno-associated virus and lentivirus. Details of these and other techniques are known in the art, for example, as described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; and Engelke, D. R., RNA Interference (RNAi): Nuts and Bolts of RNAi Technology, DNA Press LLC, Eagleville, Pa., 2003.

Suitable assays for assessment of the effects of introduction of an effective amount of an anti-PDX-1 nucleic acid compound into a cell are known in the art and illustratively include assays for PDX-1 protein and/or PDX-1 encoding RNA by one or more of: RT-PCR, Northern blot, immunoblot, immunoprecipitation, and ELISA. Additional assays for effective anti-PDX-1 nucleic acid compounds include assay of PDX-1 binding to target sequences, such as by DNA footprint analysis; assay of transcription products of genes for which PDX-1 is a transcription factor, such as by RT-PCR, Northern blot, immunoblot, immunoprecipitation, or ELISA; and assays for induction of apoptosis in cells contacted with a putative anti-PDX-1 agent, such as by assay of specific apoptosis indicators including genomic DNA fragmentation, annexin V labeling of cells, or assessment of level of active caspase-3.

In one embodiment, a method according to the present invention includes a tumor cell with an effective amount of an anti-PDX-1 agent directed to a specified region of a nucleic acid molecule encoding PDX-1 by introducing the agent into the tumor cell. At least a portion of the anti-PDX-1 agent specifically hybridizes with the nucleic acid molecule encoding PDX-1 and inhibits the expression of PDX-1 in the tumor cell. In a particular embodiment, the anti-PDX-1 agent is includes a nucleic acid sequence which is highly complementary to SEQ ID No. 3, SEQ ID No. 4, SEQ ID No. 5, or SEQ ID No. 6.

In a specific embodiment, the anti-PDX-1 agent includes an RNA sequence highly complementary to SEQ ID No. 3, SEQ ID No. 4, SEQ ID No. 5, or SEQ ID No. 6. that inhibits expression of a PDX-1 gene by RNA interference.

In a further particular embodiment, the anti-PDX-1 agent includes antisense nucleic acid sequence SEQ ID No. 8, SEQ ID No. 9, SEQ ID No. 10, or SEQ ID No. 11.

DNA sequences encoding shRNA directed to PDX-1 targets are anti-PDX-1 agents in particular embodiments of methods and compositions of the present invention. For example, DNA sequences encoding anti-PDX-1 shRNA include SEQ ID No. 22, SEQ ID No. 23 and SEQ ID No. 24.

A sequence encoding shRNA directed to control sequence (SEQ ID No. 7) may be used to observe specificity of an anti-PDX-1 agent effect. For example, the control shRNA encoded by SEQ ID No. 25 is used.

In addition, synthetic shRNA constructs are anti-PDX-1 agents in embodiments of compositions and methods of the present invention. Synthetic shRNA sequences which are anti-PDX-1 agents include SEQ ID No. 26, SEQ ID No. 27, SEQ ID No. 28 and SEQ ID No. 29. A control synthetic shRNA sequence, shControl, is SEQ ID No. 30.

### Non-Nucleic Acid Anti-PDX-1 Agents

An anti-PDX-1 agent includes peptide and protein anti-PDX-1 agents such as antibodies and peptide aptamers, as well as inhibitory compounds which are not peptides or proteins.

The term “inhibitory compound” as used herein refers to any substance, such as a molecule or complex of molecules which specifically inhibits PDX-1. Such substances illustratively include naturally occurring or synthetic small molecule chemical agents, compounds, complexes or salts thereof. Inhibitory compounds are identified by assay for a decrease in PDX-1 activity such as by assay of PDX-1 transcription, translation, transport, post-translation modification, or transcription factor function, for example.

An antibody or antibody fragment which is an anti-PDX-1 agent included in a composition and/or method of the present invention specifically binds to PDX-1 and inhibits activity of PDX-1.

The term “antibody” herein is used in its broadest sense and illustratively includes polyclonal antibodies, monoclonal antibodies, chimeric antibodies, humanized antibodies, as well as antigen binding antibody fragments and molecules having PDX-1 binding functionality. The term “antibody” includes an intact immunoglobulin having four polypeptide chains, two heavy (H) chains and two light (L) chains linked by disulfide bonds. The term antibody also includes “antigen binding antibody fragments” illustratively including such fragments as an Fab fragment, an Fab′ fragment, an F(ab′)2 fragment, an Fd fragment, an Fv fragment, an scFv fragment, and a domain antibody (dAb). In addition to uses as anti-PDX-1 agents, an antibody is optionally included in inventive compositions and methods to target an anti-PDX-1 agent to a specified location as described further below. Antibodies are generated using standard techniques, using PDX-1 or peptides corresponding to portions of PDX-1 as an antigen. Methods of antibody generation are described in detail in E. Harlow and D. Lane, Antibodies: A Laboratory Manual, Cold Spring Harbor Laboratory Press, 1988; and B. K. C. Lo, Antibody Engineering: Methods and Protocols (Methods in Molecular Biology) Humana Press, December 2003.

The term “ribozyme” refers to enzymatic RNA molecules capable of catalyzing the specific cleavage of RNA. The mechanism of ribozyme action involves sequence specific hybridization of the ribozyme molecule to complementary target RNA, followed by an endonucleolytic cleavage. A ribozyme composition includes one or more sequences complementary to PDX-1 mRNA and inhibits the expression of PDX-1.

The term “aptamer” as used herein is intended to mean a nucleic acid and/or peptide that specifically binds to a target. Aptamers are selected from pools of nucleic acids and/or peptides for their selective binding properties. In the case of a nucleic acid aptamer, the aptamer is characterized by binding interaction with a target other than Watson-Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. In the context of the present invention, an anti-PDX-1 agent which is an aptamer is characterized by PDX-1 binding and inhibition of PDX-1 function.

### Anti-PDX-1 Agent Targeting

A method of contacting the tumor cell with the anti-PDX-1 agent is achieved in certain embodiments by specific delivery of the anti-PDX-1 agent to the tumor cell. Specific delivery to a tumor cell may be achieved by a variety of delivery methods, illustratively including local injection of an anti-PDX-1 agent into a tumor and/or in the vicinity of the tumor cell. Further, an anti-PDX-1 agent is delivered in conjunction with a targeting element in particular embodiments.

A targeting element enhances delivery of an anti-PDX-1 agent across a cell barrier, to a specified tissue, to a specified cell type and/or to a specified intracellular location, for example. A wide variety of substances are useful as targeting elements for targeting an anti-PDX-1 agent in methods and compositions of the present invention, illustratively including a protein; peptide; antibody; antigen binding antibody fragment; hormone; antigen; hapten; carbohydrate binding moiety such as a lectin; enzyme; enzyme substrate; receptor; receptor ligand; substrate for a transporter, such as insulin; oligonucleotide; aptamer; and other such molecules which specifically interact with a binding partner molecule.

A targeting element which enhances delivery of an anti-PDX-1 agent to a specified intracellular location may be any of various organelle localization signals in specific embodiments. For example, a nuclear localization signal is a targeting moiety useful in enhancing localization of an anti-PDX-1 agent to a cell nucleus. Exemplary nuclear localization sequences are known in the art and are described in references such as Dingwall, C., and Laskey, R. Nuclear targeting sequences—a consensus? 1991, Trends Biochem. Sci. 16:478-481; Mattaj, I., Englmeier. Nucleocytoplasmic transport: the soluble phase. 1998, Annu. Rev. Biochem 67:265-306; Gorlich, D., Kutay, U. Transport between the cell nucleus and the cytoplasm. 1999, Annu. Rev. Cell Dev. Biol. 15:607-660; and Nakielny, S., Dreyfuss, G. Transport of proteins and RNAs in and out of the nucleus. 1999, Cell 99:677-690.

In a particular embodiment, a targeting element interacts with a marker indicative of a targeted cell. For example, particular tumors express tumor markers indicative of the status of the cell as a tumor. Such markers are known in the art, such as described in M. Fleisher (Ed.), The Clinical Biochemistry of Cancer, Washington, D.C.: American Association of Clinical Chemists, 1979; R. B. Herbman and D. W. Mercer, Eds. Immunodiagnosis of Cancer, 2nd Ed., New York: Marcel Dekker, 1990; and C. T. Garrett, S. Sell (Eds.), Cellular Cancer Markers, Humana Press, 1995. Further cells express cell-type specific markers, such as neurotransmitter receptors, peptide receptors and the like.

A targeting element is conjugated to an anti-PDX-1 agent by any of various methods. The conjugation method chosen will depend on the chemical identity of the targeting element and the anti-PDX-1 agent.

Generally, a targeting element and an anti-PDX-1 agent are linked via free functional groups on these moieties. Such functional groups illustratively include amino, carboxyl, hydroxyl, and sulfhydryl groups.

A linkage between a targeting element and an anti-PDX-1 agent is illustratively an ester, an ether, a carbamate, a carbonate, a disulfide, a peptide, and an amide. The term “linkage” refers to a bond or group formed by chemical reaction between the two moieties such that the moieties are covalently coupled, directly or indirectly.

In one embodiment, a linkage between a targeting element and an anti-PDX-1 agent is labile in an intracellular environment, such that the anti-PDX-1 agent and targeting element may be separated following cell uptake. For instance, a linkage may be susceptible to hydrolysis, enzymatic cleavage, or other form of cleavage, such that the anti-PDX-1 provides a desired effect following such separation from the targeting element. An ester linkage is one example of a linkage susceptible to hydrolysis in a cell. A disulfide linkage is a further example of a linkage susceptible to cleavage following cell uptake. In other embodiments, an anti-PDX-1 agent provides a desired effect while conjugated to the targeting element.

In one embodiment, more than one targeting element may be included in a conjugate composition. Further, more than one anti-PDX-1 agent may be included in a conjugate composition.

Where one or both of the anti-PDX-1 agent and the targeting element include a peptide and/or protein, functional group of a targeting element and an anti-PDX-1 agent used to conjugate these moieties can be at N- or C-terminus or at between the termini of one or both peptides or proteins.

A protective group may be added to an anti-PDX-1 agent and/or targeting element in a process to form a conjugate according to the present invention. Such groups, their generation and use are described in Protective Groups in Organic Synthesis by T. W. Greene and P. G. M. Wuts, John Wiley & Sons, 1999.

Conjugation chemistries used in conjugation of a targeting element and an anti-agent illustratively include coupling agents such as glutaraldehyde, carbodiimide, succinimide esters, benzidine, periodate, isothionate and combinations of these.

A targeting element and an anti-PDX-1 agent may be linked directly to form a conjugate. Alternatively, a linker may be bound to both a targeting element and to an anti-PDX-1 agent such that these moieties are indirectly linked through the linker. A linker may be a homo-bifunctional linker or a hetero-bifunctional linker, depending on the identity of the moieties to be conjugated. Further, a linker may be multifunctional so as to link more than one targeting element and/or more than one anti-PDX-1 agent.

In general, a linker has about 1-20 backbone carbon atoms. However, a linker may be larger or smaller.

A linker may be a natural or synthetic polymer in some embodiments. For example, suitable polymers include polyacrylamide, agarose, carboxymethylcellulose, cellulose, dextran, and polyaminopolystyrene.

### Compositions

A composition is provided according to the present invention which includes an anti-PDX-1 agent and a pharmaceutically acceptable carrier. A pharmaceutically acceptable carrier is substantially nontoxic to a subject and is substantially non-reactive with respect to the anti-PDX-1 agent, any included targeting element, or other ingredient.

In one embodiment, the anti-PDX-1 agent is conjugated to a cell targeting moiety.

Specific examples of inventive compositions include compositions including an anti-PDX-1 siRNA, shRNA and/or a recombinant vector including a nucleic acid encoding an siRNA, shRNA or portion thereof. In particular embodiments, an inventive composition includes an antisense nucleic acid complementary to PDX-1 mRNA. In further embodiments, an inventive composition includes an antisense nucleic acid complementary to SEQ ID No. 3, SEQ ID No. 4, SEQ ID No. 5, or SEQ ID No. 6. In preferred embodiments, an inventive composition includes an antisense nucleic acid selected from SEQ ID No. 8, SEQ ID No. 9, SEQ ID No. 10, or SEQ ID No. 11.

### Formulation and Administration of Compositions

An inventive pharmaceutical composition includes an anti-PDX-1 agent according to the present invention and a pharmaceutically acceptable carrier. The term “pharmaceutically acceptable” refers to a material which can be administered to a subject along with an inventive anti-PDX-1 agent composition without causing significant undesirable biological effects and without interacting in a deleterious manner with any other component of the pharmaceutical composition.

Pharmaceutical compositions suitable for administration illustratively include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers; diluents; solvents; or vehicles include water, ethanol, polyols such as propylene glycol, polyethylene glycol, glycerol, and the like, suitable mixtures thereof; vegetable oils such as olive oil; and injectable organic esters such as ethyloleate. Proper fluidity of liquids can be maintained, for example, by the use of a coating such as lecithin, by the maintenance of the required particle size in the case of dispersions, and by the use of surfactants.

Compositions suitable for injection optionally include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity of injectables can be maintained, for example, by the use of a coating such as lecithin, by the maintenance of the required particle size in the case of dispersions and by the use of surfactants.

Pharmaceutical compositions according to the present invention may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of an injectable pharmaceutical form can be brought about by the use of agents delaying absorption, for example, aluminum monostearate and gelatin.

Further exemplary adjuvants include immunostimulating adjuvants such as Freund's complete adjuvant; Freund's incomplete adjuvant; aluminum hydroxide such as commercially available as Alhydrogel, Accurate Chemical & Scientific Co., Westbury, N.Y.; and Gerbu adjuvant, available from C-C Biotech, Poway, Calif.

Solid dosage forms for oral administration include capsules, tablets, pills, powders, and granules. In such solid dosage forms, an inventive anti-PDX-1 agent is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol, and glycerol monostearate; h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Microencapsulated formulations of an inventive anti-PDX-1 agent are also contemplated.

Liquid dosage forms for oral administration include pharmaceutically acceptable emulsions, solutions, suspensions, syrups, and elixirs. In addition to an anti-PDX-1 agent according to the present invention, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, a pharmaceutical composition according to the present invention can also include adjuvants, such as wetting agents, emulsifying and suspending agents, sweetening, flavoring, and perfuming agents.

Suspensions, in addition to an inventive anti-PDX-1 agent, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Further specific details of pharmaceutical formulation can be found in Pharmaceutical Dosage Forms Tablets, eds. H. A. Lieberman et al. (New York: Marcel Dekker, Inc., 1989), Remington: The Science and Practice of Pharmacy, Lippincott Williams & Wilkins, 21st Ed., 2006; and in L. V. Allen, Jr. et al., Ansel's Pharmaceutical Dosage Forms and Drug Delivery Systems, 8th Ed. (Philadelphia, Pa.: Lippincott, Williams & Wilkins, 2004).

A composition including an anti-PDX-1 agent is optionally delivered in conjunction with a second therapeutic and/or diagnostic agent in one embodiment. An effective amount of a therapeutic and/or diagnostic agent is administered to achieve a therapeutic and/or diagnostic goal, illustratively including amelioration of pain, inflammation, or other signs or symptoms of a particular condition of the subject. A therapeutic and/or diagnostic agent suitable in this regard illustratively includes an analgesic, an antibiotic, an antibody, an antigen, an anti-inflammatory, an anti-tumor agent, an antiviral, a gamma or beta radiation emitting species, an enzyme, and a hormone. The anti-PDX-1 composition and second therapeutic and/or diagnostic agent may be administered together in one composition, or separately.

A conjugate of the present invention can be administered to a subject alone or as part of a pharmaceutical composition. Inventive conjugate compositions are suitable for administration to patients by a variety of systemic and local routes illustratively including intravenous, oral, parenteral, intramuscular, topical, subcutaneous and mucosal.

The dosage of an inventive pharmaceutical composition will vary based on factors such as the route of administration; the age, health, and weight of the subject to whom the composition is to be administered; the nature and extent of the subject's symptoms, if any, and the effect desired. Usually a daily dosage of an inventive conjugate is in the range of about 0.001 to 100 milligrams per kilogram of a subject's body weight. A daily dose may be administered as two or more divided doses to obtain the desired effect. An inventive pharmaceutical composition may also be formulated for sustained release to obtain desired results.

### Anti-Tumor Therapeutic Agents and Treatments

In further embodiments of inventive compositions and methods, an effective amount of an anti-tumor therapeutic agent is included. Such anti-tumor agents illustratively include chemotherapeutic agents, hormone agents, anti-angiogenic agents and immunotherapeutics. Further, a method according to the present invention optionally includes administration of an anti-tumor treatment. Illustrative anti-tumor treatments include radiation therapies.

Such drugs illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, fluorocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-I a, interferon gamma-I b, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin. An anti-cancer therapeutic may also include a pharmaceutically acceptable salt, ester, amide, hydrate, and/or prodrug of any of these or other therapeutic agents.

The term “subject” as used herein refers to humans as well as to other primates and mammals generally.

Embodiments of inventive compositions and methods are illustrated in the following examples. These examples are provided for illustrative purposes and are not considered limitations on the scope of inventive compositions and methods.

### EXAMPLES

### Example 1

### Cell Culture

Human cell lines are maintained according to standard procedures, in appropriate tissue culture mediums, supplemented with 10 to 15% fetal bovine serum (FBS), L-glutamine, penicillin (100 units/ml) and streptomycin (100 micrograms/ml) at 37° C., in a humidified atmosphere containing 5% CO₂ in the air.

### Example 2

### Human PDX-1 Small Interfering RNA (siRNA) Design

Human PDX-1 siRNA targets are designed using Dharmacon siDesign tool which builds on guidelines with eight additional criteria. Design is based on human PDX-1 mRNA sequence Gene Bank Accession number NM_{—}000209, version gi:4557672. Several candidate human PDX-1 siRNA targets with a 30-50% GC content are selected and BLAST search using NCBI BLAST is performed to identify any human PDX-1 siRNA target sequences having homology to non-PDX-1 sequences. Any such identified non-specific sequences are eliminated from consideration for use in methods or compositions according to the present invention.

Four siRNA human PDX-1 targets are used in this example:

5′ AGTTCCTATTCAACAAGTA 3′ (SEQ ID No. 3) which extends from nucleotide 590 to nucleotide 608 in the nucleotide sequence shown in SEQ ID No. 1 which encodes the full length human PDX-1 protein shown in SEQ ID No. 2.

5′ TTCCTATTCAACAAGTACA 3′ (SEQ ID No. 4) which extends from nucleotide 592 to nucleotide 610 in the nucleotide sequence shown in SEQ ID No. 1.

5′ CTTGACCGAGAGACACATC 3′ (SEQ ID No. 5) which extends from nucleotide 651 to nucleotide 669 in the nucleotide sequence shown in SEQ ID No. 1.

5′ TGACCGAGAGACACATCAA 3′ (SEQ ID No. 6) which extends from nucleotide 653 to nucleotide 671 in the nucleotide sequence shown in SEQ ID No. 1.

siControl scrambled siRNA sequences are selected which have comparable nucleotide composition as selected human PDX-1 siRNA sequences but which lack significant sequence homology to mouse, rat and human genomes. In this example, siControl sequence 5′ ACTACCGTTGTATAGGTG 3′ (SEQ ID No. 7) is used as a negative control.

The control and PDX-1 target sequences are shown as DNA sequences although it is to be understood that the target of RNA interference in a cell is an RNA molecule.

Synthetic double-stranded siRNA agents used in this example include the antisense sequences directed to PDX-1 target sequences, SEQ ID Nos. 8, 9, 10 and 11, and their complements SEQ ID No. 17, 18, 19 and 20 Control synthetic double-stranded siRNA agents include antisense sequence having SEQ ID No. 16 and the complement having SEQ ID No. 21. Each of the antisense and complementary sense sequences also included a two base 3′ overhang UU.

### Example 3

### Production of Lentivirus Constructs Encoding shRNAs

Lentivirus Gene Transfer Vector

A pLKO-1-puro expression construct is used to produce the desired lentiviral vectors in this example. FIG. 3 shows a diagrammatic representation of a pLKO-1-puro lentiviral vector and its siRNA expression cassette. Flanked by 5′ and 3′ SIN-LTR of the recombinant lentivirus vector, the siRNA expression cassette is driven by a human U6 promoter and a puromycin-resistant gene is under the control of the human hPCK promoter respectively. An insert encoding hairpin siRNA is cloned into the AgeI and EcoRI sites of the vector.

Synthetic oligonucleotides designed to encode hairpin siRNAs targeting different regions of the human PDX-1 mRNA are annealed to produce double-stranded DNA and cloned into the Age I/EcoRI sites of the pLKO-1-puro vector downstream of the human U6 promoter. For instance, oligonucleotides directed to the target sequences SEQ ID Nos. 3, 5 and 6 are synthesized for insertion into a lentivirus gene transduction plasmid. Similarly, oligonucleotides directed to the control sequence SEQ ID No. 7 are synthesized for insertion into a lentivirus gene transduction plasmid. Specific anti-PDX-1 oligonucleotides directed to the target sequences SEQ ID Nos. 3, 5 and 6 for insertion into a lentivirus gene transduction plasmid encode an antisense RNA oligonucleotide SEQ ID Nos. 8, 9, 10 or 11, and a corresponding complementary sense RNA oligonucleotide selected from SEQ ID No. 17, 18, 19 and 20. A control oligonucleotide expressed by a lentivirus encodes SEQ ID No. 16. Specific anti-PDX-1 oligonucleotides encoding anti-PDX-1 shRNAs synthesized for insertion into a lentivirus gene transduction plasmid are SEQ ID No. 22, SEQ ID No. 23 and SEQ ID No. 24. SEQ ID No. 25 encodes a control shRNA.

The resulting pLKO-1-puro constructs are transformed into competent cells, such as STBL3 chemically competent E. coli cells commercially available from INVITROGEN. The presence of the desired insert is confirmed by restriction digestion, with Mlu I and BamHI, or by PCR screening.

### Example 4

### Production of Lentiviruses

Lentiviruses are prepared by co-transfection of three plasmids into 293T cells. The plasmids are pCMV Δ8.2; the vesicular stomatitis virus, VSV-G, envelope plasmid; and the gene transfer plasmid pLKO-1-puro containing the self-inactivating LTR, respectively. The map of pLKO-1 is publicly available, for instance as described in Stewart S A et al. (RNA 2003 April; 9(4):493-501 or at http://www.stewartlab.net/PlasmidMaps/pLKO.1puro_map.pdf

Approximately 3×10⁶ 293T cells are plated in 10 cm diameter culture dishes containing 10 milliliters of media one day prior to transfection. Transfection is carried out by mixing three micrograms of lentiviral DNA encoding the desired siRNA, three micrograms of the packaging plasmid pHR′CCM8.2deltaR and pHCMV-vsvG (at a ratio of 8:1) with 18 microliters of a lipid-based transfection reagent, FUGENE 6, commercially available from Roche Molecular Biochemicals, Indianapolis, Ind., in 282 microliters of DME without serum or antibiotic for 25-30 minutes at room temperature. The relative ratio is as follows:

pLKO-1 plasmid:package plasmid (Δ8.2+VSVG)=1:1

Δ8.2: VSVG=0.899:0.111;

and the procedure is performed as detailed in the reference protocol published at http://www.stewartlab.wust1.edu/Making_Virus_{—}1.htm and in Stewart, S. A., Dykxhoorn, D. M., Palliser, D., Mizuno, H., Yu, E. Y., An, D. S., Eisen, H. N., Sabatini, D. M., Chen, I. S. Y., Hahn, W. C., Sharp, P. A., Weinberg, R. A., and Novina, C. D. Stable silencing of gene expression in primary cells by retroviral delivery of RNAi. RNA 9:493-501, 2003.

The mixture is added to the 293T cells without touching the sides of the culture dish and without change of the medium. Following 24 hours of incubation, the medium was replaced. At 48 hours post-transfection, virus is harvested and filtered through a 0.45 micron pore filter to remove any cell debris.

### Example 5

### Titration of the Vectors

To obtain an estimate of the number of transduction-competent particles obtained, puromycin resistance of cells infected with virus is examined. At least one viral particle must be present in a cell to confer puromycin resistance. NIH-3T3 cells, a mouse embryonal fibroblast cell line which does not express PDX-1 and is therefore not affected by PDX-1 siRNA are used. NIH-3T3 cells are plated at a known concentration, infected with serial dilutions of viral stock and selected with puromycin 48 hours later. The virus titer is determined by identifying the highest dilution which still protects cells from puromycin. Usually, 10⁶ effective transducing units per milliliter of producer cell conditioned medium is achieved.

### Example 6

### Transduction of Lentiviral Vectors to Target Cells

Cells are seeded in culture dishes at a density that allows achievement of 30-40% confluence in about 24 hours. Infection of target cells is carried out for four hours to overnight in the presence of 8-10 micrograms/milliliter of polybrene or protamine sulfate. After overnight culture, medium is replaced with fresh growth medium. To ensure that every cell is infected for these experiments, a second transduction is performed on the day following the first transduction, achieving a transduction efficiency close to 100%. The cancer cell lines successfully subjected to lentiviral transduction include: Pancreatic Cancer—PANC-1, UK-PANC-1, MP2; Breast Cancer—T47, MCF-7, SK-BR-3; Prostrate Cancer—PC-3, LN-CAP; and Ovarian Cancer—SK-OV-3.

### Example 7

### Western Blotting

Exponentially growing cells are lysed in RIPA lysis buffer composed of 20 millimolar Tris-HCl, pH 8.0, 100 millimolar NaCl. 1 millimolar EDTA and 1% NP-40 with a protease inhibitor cocktail. Protein concentrations in the lysates are determined using a BSA protein assay kit commercially available from Pierce, Rockford, Ill. Fifty micrograms of protein lysate from each sample is combined with 5× loading buffer composed of 312 mM Tris-HCl, pH 6.8, 50% glycerol, 10% SDS, and 0.0025% bromphenol blue. The samples are incubated at 98° C. for five minutes, incubated on ice for five minutes, and centrifuged at maximal speed in a microfuge at room temperature. Proteins are resolved on 10% SDS/polyacrylamide gels and transferred to PVDF membranes. Membranes are blocked for one hour in 5% non-fat dry milk in TBST composed of 10 mM Tris-HCL, pH 7.4, 150 mM NaCl, and 0.1% Tween 20. The blocked membranes are probed for two hours with primary antibodies diluted in TBST/5% nonfat dry milk. Membranes are then washed in TBST and incubated for one hour with appropriate horseradish peroxidase-labeled secondary antibodies. The membranes are then washed four times with TBST and antibody binding detected using chemiluminescent reagents such as ECL Western blotting reagents commercially available from Amersham. Rabbit polyclonal anti-IDX-1 (PDX-1, SFT1, IPF1—different names for the same gene) antibody are raised against synthetic peptide corresponding to the carboxyl terminal of PDX-1, reactive with PDX-1 from mouse, rat and human, purchased from Chemicon are used (catalog number AB3243).

### Example 8

### Quantitative PCR

Total RNA is prepared from cells using standard techniques (RNAEASY, Qiagen, Valencia, Calif.) and cDNA is synthesized (High Capacity cDNA Archive kit, Applied Biosystems, Foster City, Calif.). Human PDX-1 mRNA is detected using Brilliant SYBR Green detection reagents (Stratagene, La Jolla, Calif.), using the primers: 5′-AGCCGGAGGAGAACAAGC-3′ (SEQ ID No. 12) and 5′-TTCAACATGACAGCCAGCTC-3′ (SEQ ID No. 13). Amplification conditions are 10 min 95° C., followed by 40 cycles of 30 sec 95° C./60 sec 60° C. in an Mx3000P thermal cycler (Stratagene, La Jolla, Calif.). GAPDH expression was measured under the same amplification conditions as an internal control (Assays on Demand™, Applied Biosystems, Foster City, Calif.).

### Example 9

### Confirmation of Apoptosis by Annexin V Determination

Approximately 5×10⁵ cells are plated per 60 mm diameter dish and cultured overnight. Cells are then left untreated or transfected with control or anti-PDX-1 siRNA constructs. After 72 to 96 hours, cells are trypsinized, washed with 1× binding buffer, and then incubated for 15 minutes in the dark with fluorescently labeled Annexin V. Cells are then immediately analyzed by flow cytometry. The ability of cells to bind fluorescently labeled Annexin V (as monitored by the increase in the intensity of cellular fluorescence) reflects membrane changes associated with apoptosis induced by inhibition of PDX-1 expression.

### Example 10

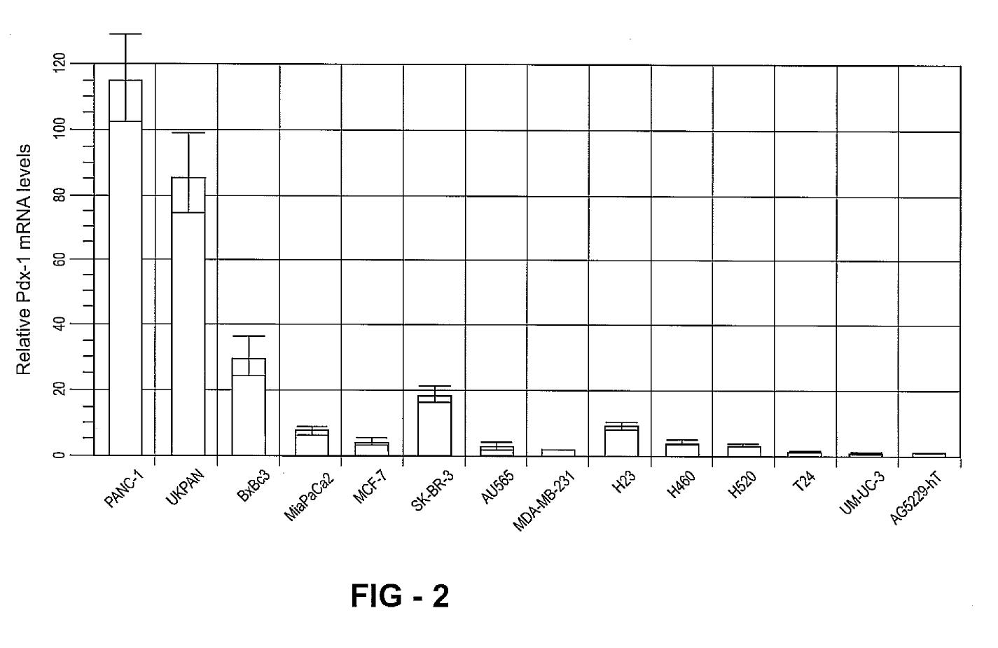

The level of PDX-1 expression in human cancer cells at the level of mRNA is presented in this example. mRNA levels in a variety of human cancer cells is quantified using quantitative real time PCR. A variety of human cancer cells are assessed, including PANC-1, UKPAN, BxBc3, MiaPaCa2, MCF-7, SK-BR-3, AU565, MDA-MB-231, H23, H460, H520, T24, Um-UC-3 and AG5229-hT. A significant proportion of these cell lines overexpress PDX-1 mRNA relative to normal human tissues excluding normal human pancreatic beta and delta cells. These results are presented in FIG. 2.

Total RNA is prepared from the cell lines indicated using standard techniques (RNAEASY, Qiagen, Valencia, Calif.) and cDNA is synthesized (High Capacity cDNA Archive kit, Applied Biosystems, Foster City, Calif.). Human PDX-1 mRNA is detected using Brilliant SYBR Green detection reagents (Stratagene, La Jolla, Calif.), using the primers: 5′-CTGGATTGGCGTTGTTTGTG-3′(SEQ ID No. 14) and 5′-CCAAGGTGGAGTGCTGTAGGA-3′ (SEQ ID No. 15). Amplification conditions are 10 min 95° C., followed by 40 cycles of 30 sec 95° C./60 sec 60° C. in an Mx3000P thermal cycler (Stratagene, La Jolla, Calif.). GAPDH expression was measured under the same amplification conditions as an internal control (Assays on Demand™, Applied Biosystems, Foster City, Calif.). Levels of PDX1 mRNA are normalized to the level of PDX-1 expressed in AG5229-hT, a normal human fibroblast cell line.

Cell lines were selected to present a panel of tumors of different tissue origin: pancreatic cancer (Panc1, BxPC3, UKPAN, MiaPaCa-2), breast cancer (MCF-7, SK-BR-3, AU 562, MDA-MB-231), lung cancer (H23, H460, H520) and bladder cancer. All of these cell lines (except AU562, which has not been investigated) can form xenograft tumors in nude mice and thus can be used to study responses in vivo.

Expression of PDX-1 in primary human tumors have been reported, as detailed in the following exemplary references. Leys C. M. et al., Expression of Pdx-1 in human gastric metaplasia and gastric adenocarcinoma. Hum Pathol. 2006 September; 37(9):1162-8. Epub 2006 Jul. 7. Wang X. P., Li Z. J., Magnusson J., Brunicardi F. C. Tissue MicroArray analyses of pancreatic duodenal homeobox-1 in human cancers. World J. Surg. 2005 March; 29(3):334-8. Koizumi M. et al., Increased PDX-1 expression is associated with outcome in patients with pancreatic cancer Surgery. 2003 August; 134(2):260-6.

### Example 11

### Inhibition of PDX-1 Expression Using Synthetic siRNAs Determined by Western Blotting

Synthetic siRNAs are used to inhibit PDX-1 expression. Approximately 5×10⁵ MiaPaCa-2 pancreatic cancer or SK-BR-3 breast cancer cells are plated in 60 mm diameter culture dishes and cultured overnight under standard conditions. Cells are then transfected with siRNA directed to SEQ ID No. 3, including oligonucleotides SEQ ID Nos. 8 and 17, or scrambled siControl siRNA directed to SEQ ID No. 7, including oligonucleotides SEQ ID Nos. 16 and 21, with Dharmafect 2 siRNA transfection reagent according to manufacturer's protocol. Each of the oligonucleotides including SEQ ID Nos. 8, 17, 16 and 21 used in this example also has a two base 3′ overhang UU. A volume of 200 microliters of 2 micromolar siRNA is used per plate for a final siRNA concentration of 100 nM. After 72-96 hours, cells are harvested and analyzed for PDX-1 expression by Western blotting. Expression of actin is determined to confirm equal protein loading.

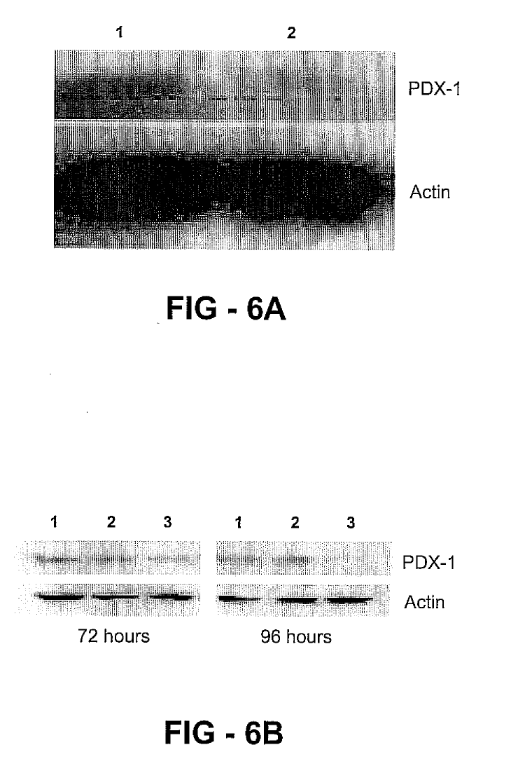

FIG. 6A is an image of Western blot demonstrating suppression of PDX-1 protein in MiaPaCa-2 pancreatic cancer cells transfected with anti-PDX-1 siRNA as opposed to control siRNA.

FIG. 6B is an image of Western blot demonstrating suppression of PDX-1 protein in SK-BR-3 breast cancer cells transfected with anti-PDX-1 siRNA compared to untransfected cells or cells transfected with control siRNA;

### Example 12

### Apoptosis in MiaPaCa2 Cells Induced by PDX-1 siRNA and Analyzed for Annexin V Binding with Flow Cytometry

Cells are plated 5×10⁵ cells/60 mm plate. After overnight incubation cells are transfected with siRNA directed to SEQ ID No. 3, including oligonucleotides SEQ ID Nos. 8 and 17, or scrambled siControl siRNA directed to SEQ ID No. 7, including oligonucleotides SEQ ID Nos. 16 and 21, with Dharmafect 2 siRNA transfection reagent according to manufacturer's protocol. Each of the oligonucleotides including SEQ ID Nos. 8, 17, 16 and 21 used in this example also has a two base 3′ overhang UU. 200 microliters of 2 micromolar siRNA is used per plate for a final siRNA concentration of 100 nM. 96 hours later cells are lifted with trypsin/EDTA, incubated with FITC-labeled Annexin V for 15 minutes in the dark and immediately analyzed by flow cytometry. The ability of cells to bind FITC-labeled Annexin V (as measured by the shift of the curve to the right) reflects membrane changes associated with apoptosis. The fraction of Annexin V-binding cells is presented in the right-hand side of each panel. FIG. 9 shows results representative of 4 experiments including these treatments. Panel A illustrates results obtained when cells are untransfected. Panel B shows results obtained when cells are transfected with siControl scrambled siRNA directed to the sequence of SEQ ID No. 7. Panel C shows the effects of transfecting cells with siRNA directed to the sequence of SEQ ID No. 3. Similar results are observed using siRNA directed to the sequence of SEQ ID No. 5.

### Example 13

### Apoptosis in Multiple Cancer Cells Induced by PDX-1 siRNA and Analyzed for Annexin V Binding with Flow Cytometry

The procedure of Example 12 is repeated in cell lines associated with breast cancer (MCF-7, T-47D, SK-BR-3), ovarian cancer (SK-OV-3) and prostrate cancer (PC3), as well as in human embryonic fibroblasts immortalized with telomerase (BJ). Cells are harvested 72 hours after transfection. The results are summarized in FIG. 10 for each of these cell Tines along with pancreatic cancer cells (MiaPaCa-2) for untransfected, siControl and siPDX1-590 (directed to target sequence SEQ ID No. 3). SiRNA directed to SEQ ID No. 3, includes antisense and sense oligonucleotides SEQ ID Nos. 8 and 17, respectively. Scrambled siControl siRNA directed to SEQ ID No: 7, includes oligonucleotides SEQ ID Nos. 16 and 21. Each of the oligonucleotides including SEQ ID Nos. 8, 17, 16 and 21 used in this example also has a two base 3′ overhang UU. Only the fibroblasts appeared refractory to PDX-1 suppression.

### Example 14

### Cell Death in Cells Treated with Synthetic siRNAs as Determined by Direct Visualization

Synthetic siRNAs are used to inhibit PDX-1 expression and induce apoptotic cell death in cultured cells. Approximately 5×10⁵ UKPAN-1, PANC-1, MiaPaCa-2 or LN-CaP (a type of human prostate cancer cell) cells are plated in 60 mm diameter culture dishes and cultured overnight under standard conditions. Cells are then transfected with 2 micromolar siRNA 600 microliters directed to SEQ ID No. 3 or scrambled siControl siRNA directed to SEQ ID No.7 using Dharmafect siRNA transfection reagent as per manufacturer's protocol. SiRNA directed to SEQ ID No. 3, includes antisense and sense oligonucleotides SEQ ID Nos. 8 and 17, respectively. Scrambled siControl siRNA directed to SEQ ID No. 7, includes oligonucleotides SEQ ID Nos. 16 and 21. Each of the oligonucleotides including SEQ ID Nos. 8, 17, 16 and 21 used in this example also has a two base 3′ overhang UU. Final siRNA concentration is 100 mM. Untreated cell cultures serve as an additional control.

Microscopic and visual examination of cultures after treatment with synthetic siRNAs to inhibit PDX-1 expression shows very nearly complete death of cells. In contrast, cells incubated with control siRNA and untreated cells show little or no cell death. If the day of siRNA transfection is day 1, then cell death is first observed around day 4 (72 hours after siRNA transfection). Almost 100% of transfected cells die by day 6.

### Example 15

### Cell Death in Cells Treated with Lentivirus-Expressed shRNAs Determined by Direct Visualization

Lentivirus-expressed shRNAs are used to inhibit PDX-1 expression and induce apoptotic cell death in cultured cells. Approximately 5×10⁵ UKPAN-1, breast cancer cells 1937 or normal mouse fibroblasts 3T3 cells are plated in 60 mm diameter culture dishes and cultured overnight under standard conditions. Cells are then incubated with lentivirus expressing shRNA directed to SEQ ID No. 3, encoded by SEQ ID No. 22, or scrambled Control shRNA directed to SEQ ID No. 7 encoded by SEQ ID No. 25, at a concentration corresponding to 20 lentivirus particles per cell. Untreated cell cultures serve as an additional control.

Microscopic and visual examination of cultures after treatment with lentiviruses expressing shRNAs to inhibit PDX-1 expression shows very nearly complete death of cells. In contrast, cells incubated with lentiviruses expressing control shRNA and untreated cells show little or no cell death. For lentivirus, if the day of the first virus infection is day 1, then apoptosis is observed around day 5 or 6 (the 4^{th} or 5^{th} day after first virus infection). Almost 100% cell death is noted by day 8 to 9. Without intending to be bound by a particular theory, it is believed that the delayed apoptosis is due to the fact that lentivirus has to first integrate into the genome before siRNA or shRNA production begins.

NIH-3T3 cells serve as a further control. Expression of anti-PDX-1 shRNAs in NIH-3T3 cells does not result in apoptosis of these cells which do not express PDX-1.

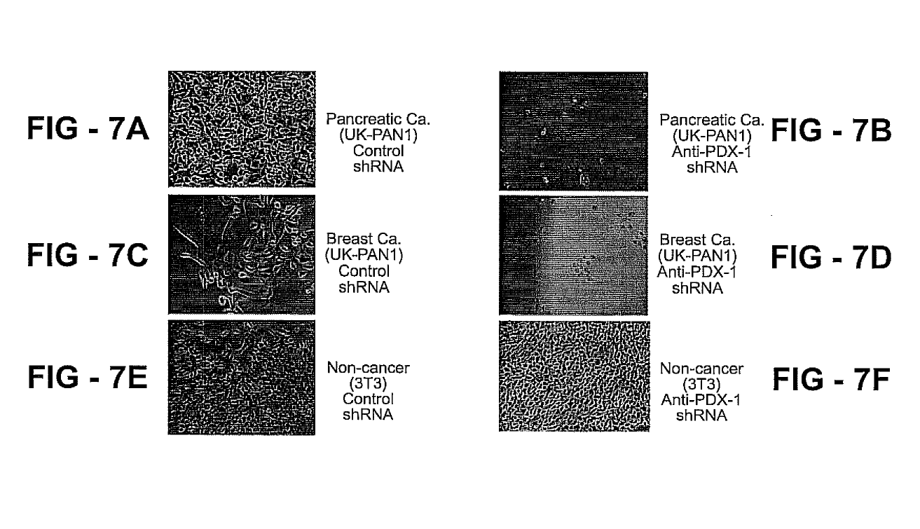

FIGS. 7A-7F show the effects of control and anti-PDX-1 shRNAs on UK-PAN-1, HCC 1937 and NIH 3T3 cells.

FIG. 7A is a digitized image illustrating pancreatic cancer cell line UK-PAN-1 cells after infection with a lentiviral vector expressing control siRNA. Comparison with FIG. 7B, a digitized image illustrating pancreatic cancer cell line UK-PAN-1 cells after infection with a lentiviral vector expressing anti-PDX-1 siRNA shows specificity of cell death in these cells.

FIG. 7C is a digitized image illustrating cells of ICC 1937 breast cancer cells after infection with a lentiviral vector expressing control siRNA. FIG. 7D is a digitized image illustrating the cell death of HCC 1937 breast cancer cells after infection with a lentiviral vector carrying an anti-PDX-1 shRNA cassette, again showing specificity of the effect of anti-PDX-1 shRNA.

FIGS. 7E and 7F show the absence of toxicity of a lantiviral vector expressing control siRNA or anti-PDX-1 shRNAs, respectively, in normal mouse embryonic fibroblast cell line 3T3.

### Example 16

### Quantitative RT-PCR Analysis of PDX-1 Expression in MiaPaCa2 Cell Transfected with Synthetic Anti-PDX-1 siRNA 590

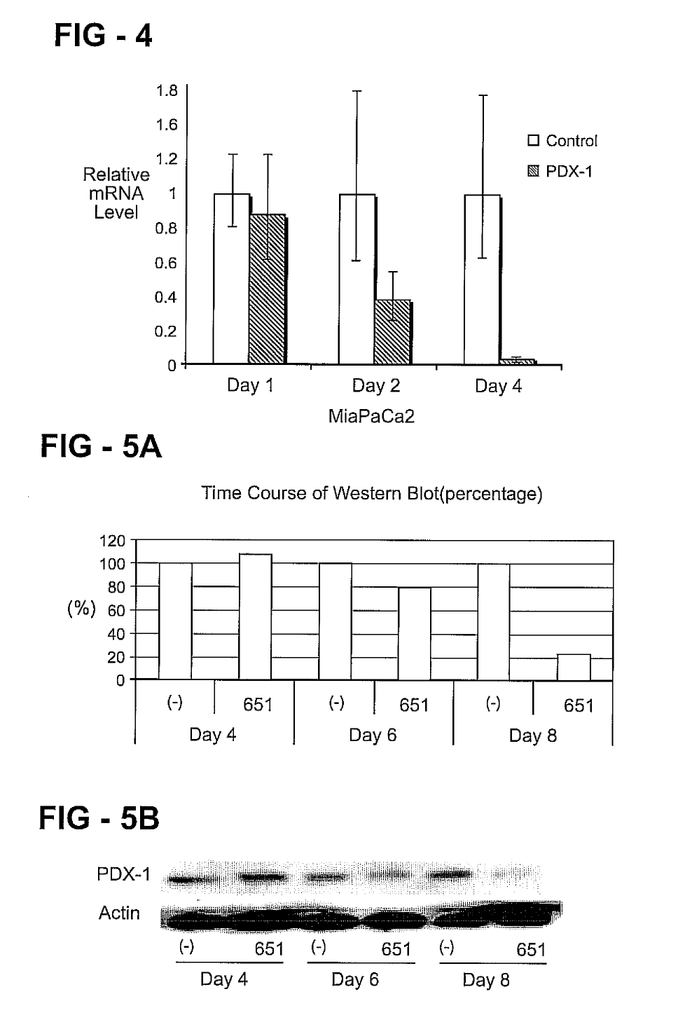

Synthetic siRNA is used to inhibit PDX-1 expression in cultured cells. Approximately 5×10⁵ MiaPaCa2 cells are plated in 60 mm diameter culture dishes and cultured overnight under standard conditions. Cells are then transfected with siRNA directed to SEQ ID No. 3, including oligonucleotides SEQ ID Nos. 8 and 17, or scrambled siControl siRNA directed to SEQ ID No. 7, including oligonucleotides SEQ ID Nos. 16 and 21, with Dharmafect 2 siRNA transfection reagent according to manufacturer's protocol. Each of the oligonucleotides including SEQ ID Nos. 8, 17, 16 and 21 used in this example also has a two base 3′ overhang UU. At 24, 48 and 96 hours after transfection, cells are lysed, total RNA is isolated, and quantitative real-time RT-PCR is performed as described in Example 8 to determine the effectiveness of the anti-PDX-1 siRNA in decreasing PDX-1 mRNA level. FIG. 4 is a graph illustrating suppression of PDX-1 mRNA in MiaPaCa2 pancreatic cancer cells transfected with anti-PDX-1 siRNA compared to cells transfected with control siRNA, as determined by quantitative real-time PCR. Level of PDX-1 RNA is normalized to the level of a housekeeping gene GAPDH in the same sample to insure equal RNA loading. Results are presented as an amount of PDX-1 RNA in cells transfected with anti-PDX-1-590 siRNA relative to cells transfected with control siRNA. There is a statistically significant decrease of the PDX-1 mRNA level on days 2 and 4.

### Example 17

### Western Blot Analysis of Pdx-1 Suppression with a Lentivirus Carrying Anti-Pdx-1 shRNA Cassette

Lentiviruses carrying shRNA cassettes are used to inhibit PDX-1 expression in cultured cells. Approximately 5×10⁵ of MiaPaCa-2 cells are plated in 60 ml diameter culture dishes and cultured overnight under standard conditions. Cells are then incubated with lentivirus carrying an shRNA cassette directed against PDX-1 target sequence SEQ ID No. 5, which is incorporated into the lentivirus using the procedures described in Example 3. DNA encoding shRNA directed to SEQ ID No. 5 is shown as SEQ ID No. 23. Cells treated with lentivirus carrying control shRNA cassette serve as negative control. DNA encoding shRNA directed to control sequence SEQ ID No. 7 is shown as SEQ ID No. 25. 4, 6 and 8 days after infection with lentiviruses, cells are lysed, total protein is isolated, and Western blot analysis is performed to determine the effectiveness of the shRNA-carrying lentiviruses in decreasing expression of PDX-1. FIG. 5A is a graph of quantified analysis of an immunoblot illustrating decreased PDX-1 levels in MiaPaCa 2 pancreatic cancer cells infected with a lentiviral vector expressing anti-PDX-1 siRNA, but not with a lentiviral vector expressing control siRNA. PDX-1 protein levels are presented relative to levels of actin.

FIG. 5B is a digitized image of an immunoblot illustrating decreased PDX-1 levels in MiaPaCa 2 pancreatic cancer cells infected with a lentivirus expressing anti-PDX-1 siRNA, but not with a lentivirus expressing control siRNA. The lower panel demonstrates equal amounts of actin in the same samples.

As demonstrated in the pictures, by day 8 after lantiviral infection expression of PDX-1 in the cells decreases to approximately 20% of control.

### Example 18

### Death of Cancer Cells Treated with Synthetic Anti-PDX-1 siRNA as Determined by Direct Visualization

Synthetic siRNA is used to inhibit PDX-1 expression and induce cell death in cultured cells. Approximately 5×10⁵ UK-PAN1 pancreatic cancer cells or LN-CAP prostate cancer cells are plated in 60 mm diameter culture dishes and cultured overnight under standard conditions. Cells are then transfected with siRNA directed to SEQ ID No. 3, including oligonucleotides SEQ ID Nos. 8 and 17, or scrambled siControl siRNA directed to SEQ ID No. 7, including oligonucleotides SEQ ID Nos. 16 and 21, with Dharmafect 2 siRNA transfection reagent according to manufacturer's protocol. Each of the oligonucleotides including SEQ ID Nos. 8, 17, 16 and 21 used in this example also has a two base 3′ overhang UU. Untreated cell cultures serve as an additional control. As shown in FIGS. 8A-8F, pancreatic and prostate cancer cells are destroyed by synthetic anti-PDX-1 siRNA, but are not harmed by synthetic control siRNA.

Nucleic Acid Sequences

| (SEQ ID No. 1) | |
|---|---|
| cgggagtggg aacgccacac agtgccaaat ccccggctcc agctcccgac tcccggctcc | 60 |
| cggctcccgg ctcccggtgc ccaatcccgg gccgcagcca tgaacggcga ggagcagtac | 120 |
| tacgcggcca cgcagcttta caaggaccca tgcgcgttcc agcgaggccc ggcgccggag | 180 |
| ttcagcgcca gcccccctga gtgcctgtac atgggccgcc agcccccgcc gccgccgccg | 240 |
| cacccgttcc ctggcgccct gggcgcgctg gagcagggca gccccccgga catctccccg | 300 |
| tacgaggtgc cccccctcgc cgacgacccc gcggtggcgc accttcacca ccacctcccg | 360 |
| gctcagctcg cgctccccca cccgcccgcc gggcccttcc cggagggagc cgagccgggc | 420 |
| gtcctggagg agcccaaccg cgtccagctg cctttcccat ggatgaagtc taccaaagct | 480 |
| cacgcgtgga aaggccagtg ggcaggcggc gcctacgctg cggagccgga ggagaacaag | 540 |
| cggacgcgca cggcctacac gcgcgcacag ctgctagagc tggagaagga gttcctattc | 600 |
| aacaagtaca tctcacggcc gcgccgggtg gagctggctg tcatgttgaa cttgaccgag | 660 |
| agacacatca agatctggtt ccaaaaccgc cgcatgaagt ggaaaaagga ggaggacaag | 720 |
| aagcgcggcg gcgggacagc tgtcgggggt ggcggggtcg cggagcctga gcaggactgc | 780 |
| gccgtgacct ccggcgagga gcttctggcg ctgccgccgc cgccgccccc cggaggtgct | 840 |
| gtgccgcccg ctgcccccgt tgccgcccga gagggccgcc tgccgcctgg ccttagcgcg | 900 |
| tcgccacagc cctccagcgt cgcgcctcgg cggccgcagg aaccacgatg agaggcagga | 960 |
| gctgctcctg gctgaggggc ttcaaccact cgccgaggag gagcagaggg cctaggagga | 1020 |
| ccccgggcgt ggaccacccg ccctggcagt tgaatggggc ggcaattgcg gggcccacct | 1080 |
| tagaccgaag gggaaaaccc gctctctcag gcgcatgtgc cagttggggc cccgcgggta | 1140 |
| gatgccggca ggccttccgg aagaaaaaga gccattggtt tttgtagtat tggggccctc | 1200 |
| ttttagtgat actggattgg cgttgtttgt ggctgttgcg cacatccctg ccctcctaca | 1260 |
| gcactccacc ttgggacctg tttagagaag ccggctcttc aaagacaatg gaaactgtac | 1320 |
| catacacatt ggaaggctcc ctaacacaca cagcggggaa gctgggccga gtaccttaat | 1380 |
| ctgccataaa gccattctta ctcgggcgac ccctttaagt ttagaaataa ttgaaaggaa | 1440 |
| atgtttgagt tttcaaagat cccgtgaaat tgatgccagt ggaatacagt gagtcctcct | 1500 |
| cttcctctgg catcaatttc acccg | 1525 |

(SEQ ID No. 2)
MNGEEQYYAATQLYKDPCAFQRGPAPEFSASPPACLYMGRQPPPPPPHPFPGALGALEQGSPPDISPYEV
PPLADDPAVAHLHHHLPAQLALPHPPAGPF PEGAEPGVLEEPNRVQLPFPWMKSTKAHAWKGQWAGGAY
AAEPEENKRTRTAYTRAQLLELEKEFLFNKYISRPRRVELAVMLNLTERHIKIWFQNRRMKWKKEEDKKR
GGGTAVGGGGVAEPEQDCAVTSGEELLALPPPPPPGGAVPPAAPVAAREGRLPPGLSASPQPSSVAPRRP
QEPR
(SEQ ID No. 3)
agttcctatt caacaagta
(SEQ ID No. 4)
ttcctattca acaagtaca
(SEQ ID No. 5)
cttgaccgag agacacatc
(SEQ ID No. 6)
tgaccgagag acacatcaa
(SEQ ID No. 7)
actaccgttg tataggtg
RNA oligonucleotide, antisense strand of siRNA directed to SEQ ID
No. 3
(SEQ ID No.8)
5′UACUUGUUGAAUAGGAACU 3′
RNA oligonucleotide, antisense strand of siRNA directed to SEQ ID
No. 4
(SEQ ID No.9)
5′ UGUACUUGUUGAAUAGGAA 3′
RNA oligonucleotide, antisense strand of siRNA directed to SEQ ID
No. 5
(SEQ ID No. 10)
5′ GAUGUGUCUCUCGGUCAAG 3′
RNA oligonucleotide, antisense strand of siRNA directed to SEQ ID
No. 6
(SEQ ID No. 11)
5′ UUGAUGUGUCUCUCGGUCA 3′
(SEQ ID No. 12)
5′-AGCCGGAGGAGAACAAGC-3′
(SEQ ID No. 13)
5′-TTCAACATGACAGCCAGCTC-3′
(SEQ ID No. 14)
5′-CTGGATTGGCGTTGTTTGTG-3′
(SEQ ID No. 15)
5′-CCAAGGTGGAGTGCTGTAGGA-3′
RNA oligonucleotide, antisense strand of siRNA directed to control
sequence SEQ ID No.7
(SEQ ID No. 16)
5′ CACCUAUACAACGGUAGU 3′
RNA oligonucleotide, sense strand of siRNA directed to SEQ ID No. 3
(SEQ ID No. 17)
5′ AGUUCCUAUUCAACAAGUA 3′
RNA oligonucleotide, sense strand of siRNA directed to SEQ ID No. 4
(SEQ ID No. 18)
5′ UUCCUAUUCAACAAGUACA 3′
RNA oligonucleotide, sense strand of siRNA directed to SEQ ID No. 5
(SEQ ID No. 19)
5′ CUUGACCGAGAGACACAUC 3′
RNA oligonucleotide, sense strand of siRNA directed to SEQ ID No. 6
(SEQ ID No. 20)
5′ UGACCGAGAGACACAUCAA 3′
RNA oligonucleotide, sense strand of siRNA directed to control
sequence SEQ ID No. 7
(SEQ ID No. 21)
5′ ACUACCGUUGUAUAGGUG 3′
DNA oligonucleotide sequence encoding shRNA directed to SEQ ID No. 3:
(SEQ ID No. 22)
5′ AGTTCCTATTCAACAAGTACTCGAGTACTTGTTGAATAGGAACT 3′
DNA oligonucleotide sequence encoding shRNA directed to SEQ ID No. 5:
(SEQ ID No. 23)
5′ CTTGACCGAGAGACACATCCTCGAGGATGTGTCTCTCGGTCAAG 3′
DNA oligonucleotide sequence encoding shRNA directed to SEQ ID No. 6:
(SEQ ID No. 24)
5′ TGACCGAGAGACACATCAACTCGAGTTGATGTGTCTCTCGGTCA 3′
DNA oligonucleotide sequence encoding shRNA directed to control
sequence (SEQ ID No. 7)
(SEQ ID No. 25)
5′ ACTACCGTTGTATAGGTGCTCGAGCACCTATACAACGGTAGT 3′
Synthetic shRNA_sequence shPDX 590 directed to SEQ ID No. 3
(SEQ ID No. 26)
5′ AGUUCCUAUUCAACAAGUACUCGAGUACUUGUUGAAUAGGAACU 3′
Synthetic shRNA sequence shPDX 592 directed to SEQ ID No. 4
(SEQ ID No. 27)
5′ UUCCUAUUCAACAAGUACACUCGAGUGUACUUGUUGAAUAGGAA 3′
Synthetic shRNA sequence shPDX 651 directed to SEQ ID No.5
(SEQ ID No. 28)
5′ CUUGACCGAGAGACACAUCCUCGAGGAUGUGUCUCUCGGUCAAG 3′
Synthetic shRNA sequence shPDX 653 directed to SEQ ID No.6
(SEQ ID No. 29)
5′ UGACCGAGAGACACAUCAACUCGAGUUGAUGUGUCUCUCGGUCA 3′
Synthetic shRNA sequence shControl directed to control sequence SEQ
ID No. 7
(SEQ ID No. 30)
5′ ACUACCGUUGUAUAGGUGGAGCUCCACCUAUACAACGGUAGU 3′

Any patents or publications mentioned in this specification are incorporated herein by reference to the same extent as if each individual publication is specifically and individually indicated to be incorporated by reference. This application claims priority of U.S. Provisional Patent Application 60/889,808 filed Feb. 14, 2007, which is incorporated herein by reference in its entirety.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of inhibiting a tumor cell, comprising:
contacting a tumor cell with an anti-PDX-1 agent, wherein the anti-PDX-1 agent inhibits PDX-1 activity, thereby inhibiting the tumor cell.

2. The method of claim 1, wherein the anti-PDX-1 agent inhibits expression of PDX-1.

3. The method of claim 1, wherein the anti-PDX-1 agent is a double-stranded RNA compound that inhibits expression of a PDX-1 gene by RNA interference.

4. The method of claim 3, wherein the double-stranded RNA compound comprises about 32 to about 60 nucleotides, an antisense strand and a sense strand, wherein the antisense strand and the sense strand each comprises about 16 to about 30 nucleotides, wherein the antisense strand comprises at least about 12 to about 26 nucleotides which are complementary to nucleotides in the sense strand and wherein the sense strand comprises at least about 12 to about 26 nucleotides which are complementary to nucleotides in the antisense strand.

5. The method of claim 1, wherein the double-stranded RNA compound is assembled from an antisense strand and a sense strand unconnected to the antisense strand.

6. The method of claim 1, wherein the double-stranded RNA compound comprises an antisense strand and a sense strand connected by a linker.

7. The method of claim 6, wherein the linker is selected from the group consisting of: an oligonucleotide linker, a polynucleotide linker and a non-nucleotide linker.

8. The method of claim 1, wherein the anti-PDX-1 agent is selected from the group consisting of: an antibody, an aptamer, and an inhibitory compound.

9. The method of claim 4, wherein the antisense strand is substantially complementary to a nucleic acid molecule encoding a human PDX-1.

10. The method of claim 4, wherein the antisense strand is substantially complementary to a nucleic acid selected from the group consisting of: SEQ ID No. 3, SEQ ID No. 4, SEQ ID No. 5, and SEQ ID No. 6.

11. The method of claim 1, wherein the anti-PDX-1 agent is selected from the group consisting of: an antisense oligonucleotide and a ribozyme.

12. A method of inhibiting PDX-1 expression in a tumor cell, comprising:
contacting a tumor cell with an effective amount of an anti-PDX-1 agent complementary to a specified region of an RNA molecule encoding PDX-1, wherein the anti-PDX-1 agent specifically hybridizes with the RNA molecule encoding PDX-1 and inhibits the expression of a PDX-1 gene in the tumor cell.

13. The method of claim 12 wherein the anti-PDX-1 agent is directed to a specified region of a nucleic acid molecule encoding human PDX-1 (SEQ ID No. 2).

14. The method of claim 12, wherein the anti-PDX-1 agent comprises an antisense nucleic acid sequence substantially complementary to a nucleic acid selected from the group consisting of: SEQ ID No. 3, SEQ ID No. 4, SEQ ID No. 5, and SEQ ID No. 6.

15. The method of claim 12, wherein the anti-PDX-1 agent is a double-stranded RNA compound that inhibits expression of the PDX-1 gene by RNA interference.

16. The method of claim 12, wherein contacting the tumor cell with the anti-PDX-1 agent is achieved by specific delivery of the anti-PDX-1 agent to the tumor cell.

17. The method of claim 16, wherein the specific delivery of the anti-PDX-1 agent to the tumor cell is mediated by a targeting element.

18. The method of claim 12 further comprising administration of a second therapeutic agent.

19. The method of claim 1, further comprising administration of an anti-cancer treatment.

20. The method of claim 1, wherein the anti-PDX-1 agent is an organic molecule inhibitor of PDX-1 expression.

21. A composition, comprising:
an anti-PDX-1 agent; and
a pharmaceutically acceptable carrier.

22. The composition of claim 21 wherein the anti-PDX-1 agent comprises an antisense oligonucleotide directed to a specified region of a nucleic acid molecule encoding PDX-1, wherein the anti-PDX-1 agent specifically hybridizes with the nucleic acid molecule encoding PDX-1 and inhibits the expression of PDX-1 in the tumor cell.

23. The composition of claim 21 wherein the anti-PDX-1 agent is conjugated to a cell targeting moiety.

24. The composition of claim 23 wherein the cell targeting moiety specifically targets the anti-PDX-1 agent to a tumor cell.

25. The composition of claim 21 further comprising a second therapeutic agent.

26. A recombinant expression construct encoding an anti-PDX-1 agent.

27. The recombinant expression construct of claim 26, encoding an antisense oligonucleotide directed to a specified region of a nucleic acid molecule encoding PDX-1, wherein the anti-PDX-1 agent specifically hybridizes with the nucleic acid molecule encoding PDX-1 and inhibits the expression of PDX-1 in the tumor cell.

28. The recombinant expression construct of claim 26 encoding an anti-PDX-1 antisense oligonucleotide selected from the group consisting of: SEQ ID No. 8, SEQ ID No. 9, SEQ ID No. 10, and SEQ ID No. 11.

29. The recombinant expression construct of claim 26 encoding a PDX-1 sense oligonucleotide selected from the group consisting of: SEQ ID No. 17, SEQ ID No. 18, SEQ ID No. 19 or SEQ ID No. 20 which in combination with an anti-PDX-1 antisense oligonucleotide selected from the group consisting of: SEQ ID No. 8, SEQ ID No. 9, SEQ ID No. 10, and SEQ ID No 11, respectively, forms a duplex RNA.

30. The recombinant expression construct of claim 28 encoding a 1-4 base 3′ overhang at the 3′ end of SEQ ID No. 8, SEQ ID No. 9, SEQ ID No. 10, or SEQ ID No. 11.

31. The recombinant expression construct of claim 29 encoding a 1-4 base 37 overhang at the 3′ end of SEQ ID No. 17, SEQ ID No. 18, SEQ ID No. 19 or SEQ ID No. 20.

32. The recombinant expression construct of claim 26 comprising an oligonucleotide encoding an anti-PDX-1 shRNA selected from the group consisting of: SEQ ID No. 22, SEQ ID No. 23 and SEQ ID No. 24.

33. The recombinant expression construct of claim 26 wherein the recombinant expression construct is a viral expression construct.


SEQUENCE LISTING (30 sequences)

SEQ ID NO: 1 - DNA, 1525 bp, Homo sapiens
cgggagtgggaacgccacacagtgccaaatccccggctccagctcccgactcccggctcccggctcccggctcccggtgcccaatcccgggccgcagccatgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagccccccggacatctccccgtacgaggtgccccccctcgccgacgaccccgcggtggcgcaccttcaccaccacctcccggctcagctcgcgctcccccacccgcccgccgggcccttcccggagggagccgagccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacgatgagaggcaggagctgctcctggctgaggggcttcaaccactcgccgaggaggagcagagggcctaggaggaccccgggcgtggaccacccgccctggcagttgaatggggcggcaattgcggggcccaccttagaccgaaggggaaaacccgctctctcaggcgcatgtgccagttggggccccgcgggtagatgccggcaggccttccggaagaaaaagagccattggtttttgtagtattggggccctcttttagtgatactggattggcgttgtttgtggctgttgcgcacatccctgccctcctacagcactccaccttgggacctgtttagagaagccggctcttcaaagacaatggaaactgtaccatacacattggaaggctccctaacacacacagcggggaagctgggccgagtaccttaatctgccataaagccattcttactcgggcgacccctttaagtttagaaataattgaaaggaaatgtttgagttttcaaagatcccgtgaaattgatgccagtggaatacagtgagtcctcctcttcctctggcatcaatttcacccg

SEQ ID NO: 2 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 3 - DNA, 19 bp, Artificial Sequence
  target sequence
agttcctattcaacaagta

SEQ ID NO: 4 - DNA, 19 bp, Artificial Sequence
  target sequence
ttcctattcaacaagtaca

SEQ ID NO: 5 - DNA, 19 bp, Artificial Sequence
  target sequence
cttgaccgagagacacatc

SEQ ID NO: 6 - DNA, 19 bp, Artificial Sequence
  target sequence
tgaccgagagacacatcaa

SEQ ID NO: 7 - DNA, 18 bp, Artificial Sequence
  target sequence
actaccgttgtataggtg

SEQ ID NO: 8 - RNA, 19 nt, Artificial Sequence
  antisense strand of siRNA directed to SEQ ID NO 3
uacuuguugaauaggaacu

SEQ ID NO: 9 - RNA, 19 nt, Artificial Sequence
  antisense strand of siRNA directed to SEQ ID NO 4
uguacuuguugaauaggaa

SEQ ID NO: 10 - RNA, 19 nt, Artificial Sequence
  antisense strand of siRNA directed to SEQ ID NO 5
gaugugucucucggucaag

SEQ ID NO: 11 - RNA, 19 nt, Artificial Sequence
  antisense strand of siRNA directed to SEQ ID NO 6
uugaugugucucucgguca

SEQ ID NO: 12 - DNA, 18 bp, Artificial Sequence
  primer
agccggaggagaacaagc

SEQ ID NO: 13 - DNA, 20 bp, Artificial Sequence
  primer
ttcaacatgacagccagctc

SEQ ID NO: 14 - DNA, 20 bp, Artificial Sequence
  primer
ctggattggcgttgtttgtg

SEQ ID NO: 15 - DNA, 21 bp, Artificial Sequence
  primer
ccaaggtggagtgctgtagga

SEQ ID NO: 16 - RNA, 18 nt, Artificial Sequence
  antisense strand of siRNA directed to control sequence SEQ ID NO 7
caccuauacaacgguagu

SEQ ID NO: 17 - RNA, 19 nt, Artificial Sequence
  sense strand of siRNA directed to SEQ ID NO 3
aguuccuauucaacaagua

SEQ ID NO: 18 - RNA, 19 nt, Artificial Sequence
  sense strand of siRNA directed to SEQ ID NO 4
uuccuauucaacaaguaca

SEQ ID NO: 19 - RNA, 19 nt, Artificial Sequence
  sense strand of siRNA directed to SEQ ID NO 5
cuugaccgagagacacauc

SEQ ID NO: 20 - RNA, 19 nt, Artificial Sequence
  sense strand of siRNA directed to SEQ ID NO 6
ugaccgagagacacaucaa

SEQ ID NO: 21 - RNA, 18 nt, Artificial Sequence
  sense strand of siRNA directed to control sequence SEQ ID NO 7
acuaccguuguauaggug

SEQ ID NO: 22 - DNA, 44 bp, Artificial Sequence
  shRNA directed to SEQ ID NO 3
agttcctattcaacaagtactcgagtacttgttgaataggaact

SEQ ID NO: 23 - DNA, 44 bp, Artificial Sequence
  shRNA directed to SEQ ID NO 5
cttgaccgagagacacatcctcgaggatgtgtctctcggtcaag

SEQ ID NO: 24 - DNA, 44 bp, Artificial Sequence
  shRNA directed to SEQ ID NO 6
tgaccgagagacacatcaactcgagttgatgtgtctctcggtca

SEQ ID NO: 25 - DNA, 42 bp, Artificial Sequence
  shRNA directed to control sequence (SEQ ID NO 7)
actaccgttgtataggtgctcgagcacctatacaacggtagt

SEQ ID NO: 26 - RNA, 44 nt, Artificial Sequence
  shRNA sequence shPDX 590 directed to SEQ ID NO 3
aguuccuauucaacaaguacucgaguacuuguugaauaggaacu

SEQ ID NO: 27 - RNA, 44 nt, Artificial Sequence
  shRNA sequence shPDX 592 directed to SEQ ID NO 4
uuccuauucaacaaguacacucgaguguacuuguugaauaggaa

SEQ ID NO: 28 - RNA, 44 nt, Artificial Sequence
  shRNA sequence shPDX 651 directed to SEQ ID NO 5
cuugaccgagagacacauccucgaggaugugucucucggucaag

SEQ ID NO: 29 - RNA, 44 nt, Artificial Sequence
  shRNA sequence shPDX 653 directed to SEQ ID NO 6
ugaccgagagacacaucaacucgaguugaugugucucucgguca

SEQ ID NO: 30 - RNA, 42 nt, Artificial Sequence
  shRNA sequence shControl directed to control sequence SEQ ID NO 7
acuaccguuguauagguggagcuccaccuauacaacgguagu